(19) United States
(12) Patent Application Publication
(10) Pub. No.: US 2022/0033426 A1
(43) Pub. Date: Feb. 3, 2022

(54) Transition Metal Macrocyclics as MRI Contrast Agents for Molecular Imaging

(71) Applicant: YALE UNIVERSITY, New Haven, CT (US)

(72) Inventors: A B M Zakaria, Danbury, CT (US); Fahmeed Hyder, Cheshire, CT (US); Samuel Maritim, Cambridge, MA (US); Jelena Milhailovic, New Haven, CT (US); Daniel Coman, New Haven, MA (US)

(21) Appl. No.: 17/387,258

(22) Filed: Jul. 28, 2021

Related U.S. Application Data
(60) Provisional application No. 63/057,359, filed on Jul. 28, 2020.

(51) Int. Cl. C07F 15/06; C07F 15/04; C07F 15/02; A61K 49/10; A61K 31/436; A61K 31/44; A61K 39/395; A61K 31/17; A61K 31/495; A61P 35/00

(52) U.S. Cl. CPC C07F 15/065; C07F 15/045; C07F 15/025; A61K 49/106; A61P 35/00; A61K 31/44; A61K 39/3955; A61K 31/17; A61K 31/495; A61K 31/436

(57) ABSTRACT

In one aspect, the present disclosure relates to a Magnetic Resonance Imaging (MRI) and Spectroscopic Imaging (MRSI) agent wherein the agent comprises a polyazamacrocyclic ligand coordinated to a first row transition metal ion. In another aspect, the disclosure relates to a method of using the MRI/MRSI agents of the present disclosure to monitor tissue temperature and/or pH in a patient in need thereof. In another aspect, the disclosure relates to a method of using the MRI/MRSI agents of the present disclosure to monitor the efficacy of a cancer treatment in a patient in need thereof.

### CROSS-REFERENCE TO RELATED APPLICATION

This application claims the benefit of priority to U.S. Provisional Patent Application Ser. No. 63/057,359 entitled “TRANSITION METAL MACROCYCLICS AS MRI CONTRAST AGENTS FOR MOLECULAR IMAGING,” filed Jul. 28, 2020, the disclosure of which is incorporated herein by reference in its entirety.

### STATEMENT REGARDING FEDERALLY SPONSORED RESEARCH

This invention was made with government support under EB023366 awarded by National Institutes of Health. The government has certain rights in the invention.

### BACKGROUND

Magnetic Resonance Imaging (MRI) and Spectroscopic Imaging (MRSI) can provide diagnostics, detection and/or valuable information for certain underlying mechanisms of diseases and therapeutic treatments. Among many biomarkers of diseases, temperature and pH are the two important biochemical parameters for tracking pathological conditions, because the human body actively regulates pH and temperature in order to meet the metabolic demands and to maintain normal healthy physiological conditions. Moreover, local variations from the tightly regulated temperature and pH can lead to heterogeneous blood flow and nutrient supply which might alter the physiological states as well as induce various cancers. For example, extracellular acidosis (i.e. low pHe) is a tumor microenvironment hallmark, caused by atypical metabolism and perfusion. Acidic pHe enhances cancer growth, proliferation, and builds therapy resistance. However, conventional MRI methods are insensitive to physicochemical parameters like pHe and mainly track intratumoral volume.

Among the primary MRI methods are paramagnetic agents for longitudinal (T₁) contrast, where assessment of treatment response involves 2D or 3D measurement with Gd³⁺ enhanced MRI contrast. These MRI methods are not reliable in distinguishing pseudoprogression and pseudoresponse from actual changes in tumor status. Because acidic pHe milieu is conducive to tumor growth and builds resistance to therapies, simultaneous mapping of pHe inside and outside the tumor (i.e. intratumoral-peritumoral pHe gradient) is an important cancer imaging need.

Several MRI and MRSI methods are available to monitor tissue temperature and pH using either endogenous and exogeneous agents. For example, ³¹P-MRSI signals of endogenous inorganic phosphate (Pi) and phosphocreatine (PCr) and exogenous agents, such as, 3-aminopropylphosphate have been used for pH (extra- and intracellular) measurement. ¹⁹F-MRSI methods using exogenous fluorine-based agents, such as, fluorinated derivatives of vitamin B6, DOTA (tetraazacyclododecane-1,4,7,10-tetraacetic acid) exploit pH-sensitive fluorine chemical shifts to measure tissue pH. An MRI method named Chemical Exchange Saturation Transfer (CEST) uses the exchangeable proton of either endogenous amine (—NHₓ), or hydroxyl (—OH) groups of intracellular proteins and peptides or exogenous chelates containing paramagnetic ions (lanthanide ions, e.g., Gd³⁺, Eu³⁺, Yb³⁺, Tm³⁺) for temperature and pH measurements. However, these MRI/MRSI methods and the corresponding agents have some inherent limitations. For example, the ³¹P-MRSI method for pH determination has relatively low sensitivity, while the fluorinated compounds used for ¹⁹F-MRSI methods are relatively unstable and require installation of fluorine coils on clinical scanners. CEST methods are limited by the high concentrations required for the probe, the effect of endogenous magnetization transfer and the requirement for high saturation power.

There still remains a need in the art for novel MRI/MRSI agents that are sensitive to changes in temperature and/or pH. There is also a need in the art for a novel method of monitoring changes in the tissue temperature and/or pH in a patient using an MRI/MRSI agent. The present invention satisfies these unmet needs.

### BRIEF SUMMARY OF THE INVENTION

In various aspects, a compound of Formula Ia or Formula IIa is provided herein:

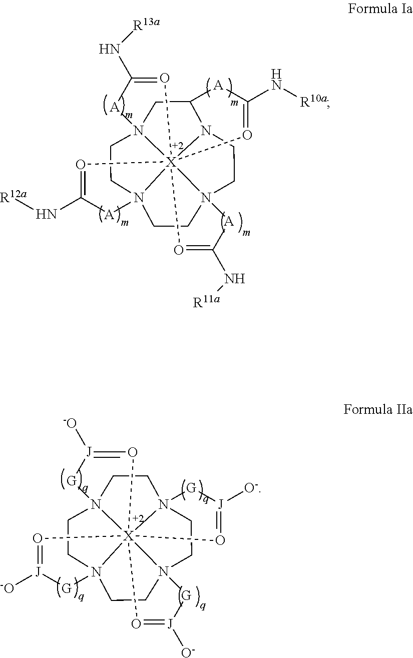

In the compounds of Formula Ia or IIa

each of R^{10a}, R^{11a}, R^{12a}, and R^{13a} is independently selected from the group consisting of

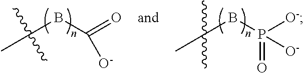

each of A and B is independently CR^{14a}R^{15a};

each G is independently CR^{20a}R^{21a};

each J is independently C or P(O⁻);

each of R^{14a} and R^{15a} is independently selected from the group consisting of hydrogen, C₁-C₆ linear alkyl, and C₃-C₆ branched alkyl, wherein adjacent R^{14a} and R^{15a} groups may bond or fuse to form a C₃-C₁₂ cycloalkyl;

each of R^{20a} and R^{21a} is independently selected from the group consisting of hydrogen, C₁-C₆ linear alkyl, and C₃-C₆ branched alkyl, wherein adjacent R^{20a} and R^{21a} groups may bond or fuse to form a C₃-C₁₂ cycloalkyl;

X is a transition metal selected from the group consisting of Fe, Zn, Co, and Ni, Mn, Au, Pt, Cr, Pd, and Ti; and

each of m, n, and q is independently an integer from 1 to 6.

In various aspects, compounds of Formula Ia and IIa are useful MRI/MRSI agents that in various aspects are sensitive to changes in temperature and/or pH.

The compounds of Formula Ia and Ha, in various aspects, can be used in a method of monitoring at least one of tissue temperature and pH in a patient in need thereof by administering these compounds to a patient. Additionally, compounds of Formula Ia and Ha can be used in a method of monitoring the efficacy of a cancer treatment in a patient in need thereof, which in various aspects includes administering to a patient a chemotherapy drug or pharmaceutical agent and administering to the patient, at the site of a tumor, a compound of Formula Ia or Formula IIa.

### BRIEF DESCRIPTION OF THE DRAWINGS

The following detailed description of exemplary embodiments of the invention will be better understood when read in conjunction with the appended drawings. For the purpose of illustrating the invention, non-limiting embodiments are shown in the drawings. It should be understood, however, that the invention is not limited to the precise arrangements and instrumentalities of the embodiments shown in the drawings.

FIG. 1 depicts an illustrative ¹H NMR spectrum of [FeDOTA-4AmC]²⁻.

FIG. 2 depicts illustrative ¹H NMR spectra of the [XDOTMA]²⁻ complexes with the assignment of non-exchangeable protons (H2, H3, and —CH₃).

FIG. 3 depicts an illustrative ¹H NMR spectrum of [FeDOTA]²⁻.

FIG. 4 depicts illustrative ¹H NMR spectra of the [XDOTA-4AmC]²⁻ complexes with the assignment of the amide proton (—NH).

FIG. 5 depicts illustrative ¹H NMR spectra of the [XDOTA-4AmP]²⁻ complexes with the assignment of the amide proton (—NH).

FIG. 6 depicts illustrative ¹H NMR spectra of a [NiDOTA-4AmC]²⁻ complex at variable temperature (25 to 42° C.).

FIG. 7 depicts illustrative ¹H NMR spectra of a [NiDOTA-4AmC]²⁻ complex at variable pH (6.5 to 8.0).

FIGS. 8A-8C depict 3D surface plots showing illustrative temperature and pH dependencies of [XDOTA-4AmC]²⁻ MRI/MRSI agents. FIG. 8A is a plot showing illustrative N—H proton chemical shifts of [FeDOTA-4AmC]²⁻. FIG. 8B is a plot showing illustrative N—H proton chemical shifts of [CoDOTA-4AmC]²⁻. FIG. 8C is a plot showing illustrative N—H proton chemical shifts of [NiDOTA-4AmC]²⁻.

FIGS. 9A-9C depict 3D surface plots showing illustrative temperature and pH dependencies of [XDOTA-4AmP]⁶⁻ MRI/MRSI agents. FIG. 9A is a plot showing illustrative N H proton chemical shifts of [FeDOTA-4AmP]⁶⁻. FIG. 9B is a plot showing illustrative N—H proton chemical shifts of [CoDOTA-4AmP]⁶⁻. FIG. 9C is a plot showing illustrative N—H proton chemical shifts of [NiDOTA-4AmP]⁶⁻.

FIGS. 10A-10C depict 3D surface plots showing illustrative temperature and pH dependencies of [XDOTMA]²⁻ MRI/MRSI agents. FIG. 10A is a plot showing illustrative —CH₃ proton chemical shifts of [FeDOTMA]²⁻. FIG. 10B is a plot showing illustrative —CH₃ proton chemical shifts of [CoDOTMA]²⁻. FIG. 10C is a plot showing illustrative —CH₃ proton chemical shifts of [NiDOTMA]²⁻.

### DETAILED DESCRIPTION OF THE DISCLOSURE

The present disclosure provides in one aspect MRI/MRSI agents comprising a polyazamacrocyclic ligand coordinated to a transition metal ion. In certain embodiments, the transitional metal ion is an ion of a first row transition metal. In some embodiments, the polyazamacrocyclic ligand comprises a proton which is sensitive to changes in pH and/or temperature. In yet other embodiments, the MRI/MRSI agents of the disclosure can be used to monitor tissue temperature and/or pH in a patient in need thereof. In some embodiments, the tissue temperature and/or pH is monitored by measuring a change in the chemical shift of one or more protons in the MRI/MRSI agent. In yet other embodiments, the MRI/MRSI agents of the disclosure can be used to monitor the efficacy of a cancer treatment in a patient in need thereof. In some embodiments, the efficacy of a cancer treatment is monitored by measuring a change in the chemical shift of one or more protons in the MRI/MRSI agent.

Most of the MRI or MRSI methods for measuring temperature and pH utilize exogenous agents composed of paramagnetic lanthanide ion (e.g. Gd³⁺, Eu³⁺, Yb³⁺, Tm³⁺) complexes. MRI/MRSI contrast agents based on endogenous first row transition metals remain undeveloped, despite the possibility that such transition metal complexes are better tolerated by animals or humans and could provide a more bio-compatible alternative to the lanthanide ions. As shown elsewhere herein, a series of MRI/MRSI agents were developed comprising first row transition metal ions complexed with polyazamacrocyclic ligands having protons which are sensitive to pH and/or temperature. In certain embodiments, the chemical shift variations of these protons can be used to detect changes in pH and/or temperature with the BIRDS (Biosensor Imaging of Redundant Deviation Shifts) method.

The skilled artisan will understand that the disclosure is not limited to the MRI/MRSI agents discussed herein. Further, the skilled artisan will understand that the MRI/MRSI agents can be administered to a patient alone or in combination with other MRI/MRSI agents. Still further, a skilled artisan will understand that the MRI/MRSI agents can be administered to a patient after the patient has received a therapeutic treatment in order to monitor changes in tissue pH and/or temperature following the therapeutic treatment. Still further, a skilled artisan will understand that the MRI/MRSI agents can be administered to a patient after treatment with a chemotherapy drug or pharmaceutical agent in order to monitor the efficacy of the treatment.

### Definitions

Unless defined otherwise, all technical and scientific terms used herein have the same meaning as commonly understood by one of ordinary skill in the art to which this disclosure pertains. Although any methods and materials similar or equivalent to those described herein can be used in the practice or testing of the present invention, selected methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compounds of Formula Ia or Ha described herein, and are physiologically acceptable to the patient. Supplementary active compounds may also be incorporated into the compositions. The “pharmaceutically acceptable carrier” may further include a pharmaceutically acceptable salt of the compounds of Formula Ia or IIa described herein. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject, or individual is a human.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the disclosure (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some instances, hyperproliferative disorders are referred to as a type of cancer including but not limited to primary or metastatic melanoma, thymoma, lymphoma, sarcoma, lung cancer, liver cancer, non-Hodgkin's lymphoma, Hodgkins lymphoma, leukemias, uterine cancer, cervical cancer, bladder cancer, kidney cancer and adenocarcinomas such as breast cancer, prostate cancer, ovarian cancer, pancreatic cancer, and the like.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, particularly ethyl, methyl, isopropyl, isobutyl, n-pentyl, n-hexyl and cyclopropylmethyl.

As used herein, the term “cycloalkyl” refers to a mono cyclic or polycyclic non-aromatic radical, wherein each of the atoms forming the ring (i.e. skeletal atoms) is a carbon atom. In certain embodiments, the cycloalkyl group is saturated or partially unsaturated. In other embodiments, the cycloalkyl group is fused with an aromatic ring. Cycloalkyl groups include groups having from 3 to 10 ring atoms. Illustrative examples of cycloalkyl groups include, but are not limited to, the following moieties:

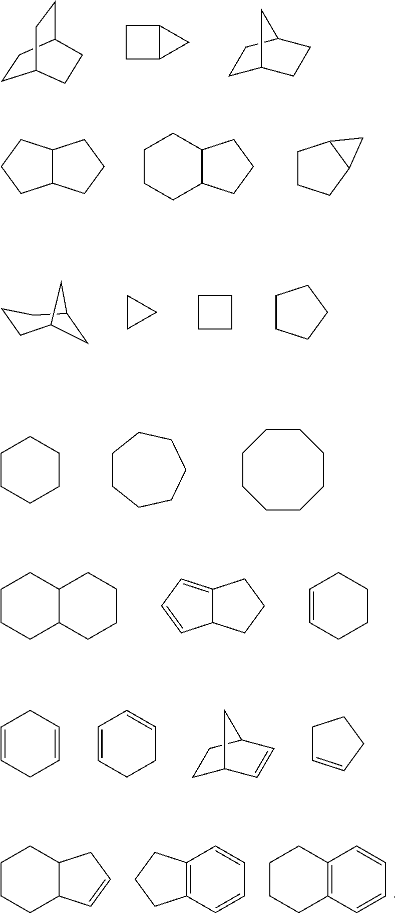

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

### Compounds and Compositions

In one aspect, the present disclosure relates to an MRI/MRSI agent comprising a polyazamacrocyclic ligand coordinated to a transition metal ion. In certain embodiments, the MRI/MRSI agent is an MRI/MRSI contrast agent. In certain embodiments, the transition metal ion is a paramagnetic transition metal ion. The transition metal ion may be an ion of any transition metal known to a person of skill in the art. Exemplary transition metals include, but are not limited to, scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, and mercury. In some embodiments, the transition metal is a first row transition metal. In some embodiments, the transition metal is selected from the group consisting of iron, zinc, cobalt, nickel, copper, manganese, gold, platinum, chromium, palladium, and titanium. In some embodiments, the polyazamacrocyclic ligand is coordinated to a Fe²⁺, Co²⁺, or Ni²⁺ transition metal ion.

The polyazamacrocyclic ligand may be any polyazamacrocyclic ligand known to a person of skill in the art. In certain embodiments, the polyazamacrocylic ligand comprises a functional group used for pH sensing, temperature sensing, or the combination of pH and temperature sensing. Exemplary functional groups used for pH and/or temperature sensing include, but are not limited to, a phosphonate group, a carboxylate group, an amide group, a hydroxy group, pyridine, benzimidazole, pyrazole, an alkyl group, or combinations thereof. In certain embodiments, the polyazamacrocyclic ligand comprises both a phosphonate group and an amide group. In other embodiments, the polyazamacrocyclic ligand comprises both a carboxylate group and an amide group. In certain embodiments, the polyazamacrocyclic ligand comprises both a carboxylate group and an alkyl group. In certain embodiments, the alkyl group is a C₁-C₆ linear alkyl. In certain embodiments, the alkyl group is methyl.

In certain embodiments, the polyazamacrocyclic ligand is a tetraazacyclododecane-1,4,7,10-tetraacetic acid (DOTA) derivative. In other embodiments, the polyazamacrocyclic ligand is a tetramethyl-1,4,7,10-tetraazacyclododecane-1,4,7,10-tetraacetic acid (DOTMA) derivative.

In certain embodiments, the polyazamacrocyclic ligand is a ligand of Formula I:

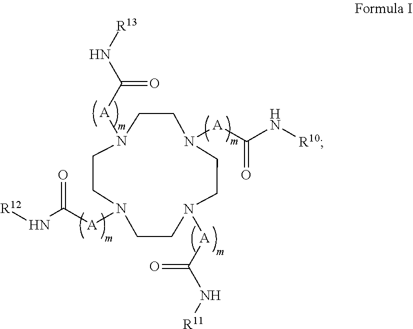

wherein

each of R¹⁰, R¹¹, R¹², and R¹³ is independently selected from the group consisting of

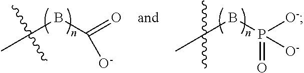

each of A and B is independently CR¹⁴R¹⁵;

each of R¹⁴ and R¹⁵ is independently selected from the group consisting of hydrogen, C₁-C₆ linear alkyl, and C₃-C₆ branched alkyl, wherein adjacent R¹⁴ and R¹⁵ groups may bond or fuse to form a C₃-C₁₂ cycloalkyl; and

each of m and n is independently an integer from 1-6.

In certain embodiments, each of R¹⁰, R¹¹, R¹², and R¹³ is independently

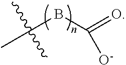

In certain embodiments, each of R¹⁰, R¹¹, R¹², and R¹³ is independently

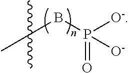

In certain embodiments, each m is 1.

In certain embodiments, each n is 1.

In certain embodiments, each of R¹⁴ and R¹⁵ is hydrogen.

In certain embodiments, at least one of R¹⁴ and R¹⁵ is a C₁-C₆ linear alkyl. In certain embodiments, each instance of R¹⁴ is a C₁-C₆ linear alkyl. In certain embodiments, each instance of R¹⁴ is methyl. In certain embodiments, each instance of R¹⁵ is a C₁-C₆ linear alkyl. In certain embodiments, each instance of R¹⁵ is methyl.

In certain embodiments, the ligand of Formula I is

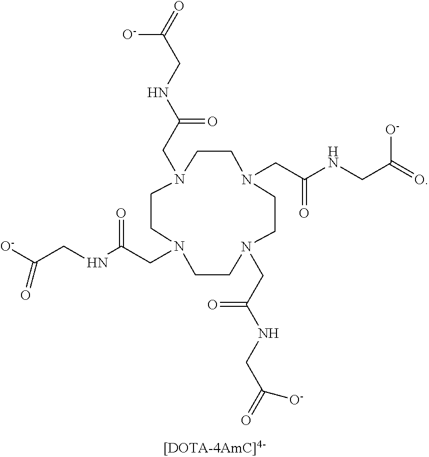 [SMILES: O=C([O-])CNC(=O)CN1CCN(CC(=O)NCC(=O)[O-])CCN(CC(=O)NCC(=O)[O-])CCN(CC(=O)NCC(=O)[O-])CC1]

In certain embodiments, the ligand of Formula I is

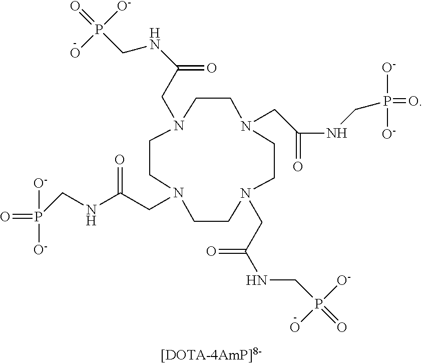 [SMILES: O=C(CN1CCN(CC(=O)NCP(=O)([O-])O)CCN(CC(=O)NCP(=O)([O-])[O-])CCN(CC(=O)NCP(=O)([O-])O)CC1)NCP(=O)([O-])[O-]]

In other embodiments, the polyazamacrocyclic ligand is a ligand of Formula II:

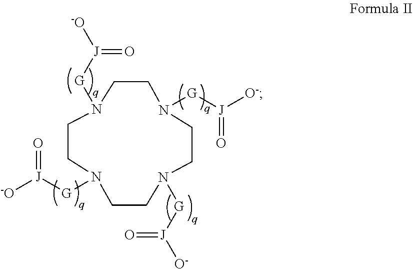

wherein:

each G is independently CR²⁰R²¹;

each J is independently C or P(O⁻);

each of R²⁰ and R²¹ is independently selected from the group consisting of hydrogen, C₁-C₆ linear alkyl, and C₃-C₆ branched alkyl, wherein adjacent R²⁰ and R²¹ groups may bond or fuse to form a C₃-C₁₂ cycloalkyl; and

each q is independently an integer from 1-6.

In certain embodiments, each of J is C.

In certain embodiments, each J is P(O⁻).

In certain embodiments, each q is 1.

In certain embodiments, each of R²⁰ and R²¹ is hydrogen.

In certain embodiments, at least one of R²⁰ and R²¹ is a C₁-C₆ linear alkyl. In certain embodiments, each instance of R²⁰ is a C₁-C₆ linear alkyl. In certain embodiments, each instance of R²⁰ is methyl. In certain embodiments, each instance of R²¹ is a C₁-C₆ linear alkyl. In certain embodiments, each instance of R²¹ is methyl.

In certain embodiments, the ligand of Formula II is

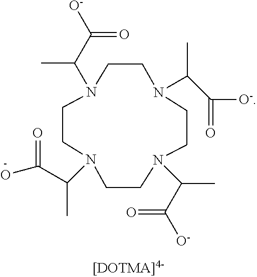

In certain embodiments, the MRI/MRSI agent comprising a ligand of Formula I is an MRI/MRSI agent of Formula Ia:

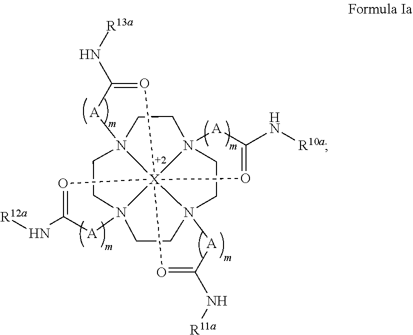

wherein

each of R^{10a}, R^{11a}, R^{12a}, and R^{13a} is independently selected from the group consisting of

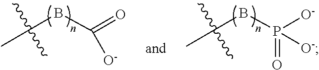

each of A and B is independently CR^{14a}R^{15a};

each of R^{14a} and R^{15a} is independently selected from the group consisting of hydrogen, C₁-C₆ linear alkyl, and C₃-C₆ branched alkyl, wherein adjacent R^{14a} and R^{15a} groups may bond or fuse to form a C₃-C₁₂ cycloalkyl;

X is a transition metal selected from the group consisting of Fe, Zn, Co, Ni, Mn, Au, Pt, Cr, Pd, and Ti; and

each of m and n is independently an integer from 1-6.

In certain embodiments, each of R^{10a}, R^{11a}, R^{12a}, and R^{13a} is

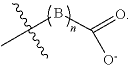

In certain embodiments, each of R^{10a}, R^{11a}, R^{12a}, and R^{13a} is

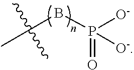

In certain embodiments, each m is 1.

In certain embodiments, each n is 1.

In certain embodiments, each of R^{14a} and R^{15a} is hydrogen.

In certain embodiments, at least one of R^{14a} and R^{15a} is a C₁-C₆ linear alkyl. In certain embodiments, each instance of R^{14a} is a C₁-C₆ linear alkyl. In certain embodiments, each instance of R^{14a} is methyl. In certain embodiments, each instance of R^{15a} is a C₁-C₆ linear alkyl. In certain embodiments, each instance of R^{15a} is methyl.

In certain embodiments, X is Fe. In other embodiments, X is Co. In other embodiments, X is Ni.
In certain embodiments, the MRI/MRSI agent of Formula Ia is

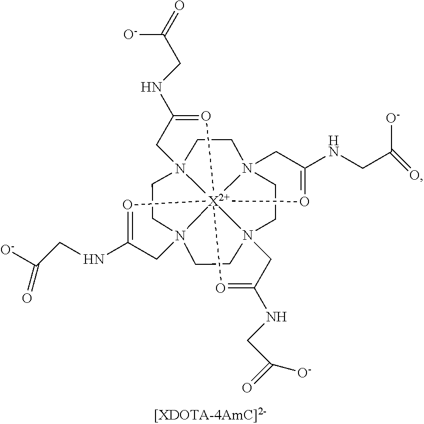

wherein X is selected from the group consisting of Fe, Zn, Co, Ni, Mn, Au, Pt, Cr, Pd, and Ti. In certain embodiments, the MRI/MRSI agent of Formula Ia is [XDOTA-4AmC]²⁻ wherein X is Fe. In other embodiments, the MRI/MRSI agent of Formula Ia is [XDOTA-4AmC]²⁻ wherein X is Co. In certain embodiments, the MRI/MRSI agent of Formula Ia is [XDOTA-4AmC]²⁻ wherein X is Ni. In certain embodiments, [XDOTA-4AmC]²⁻ exhibits high chemical shift sensitivity to temperature and pH. In certain embodiments, the exchangeable amide protons are sensitive to changes in pH and temperature. In certain embodiments, the carboxylate groups are sensitive to changes in pH.

In certain embodiments, the MRI/MRSI agent of Formula Ia is

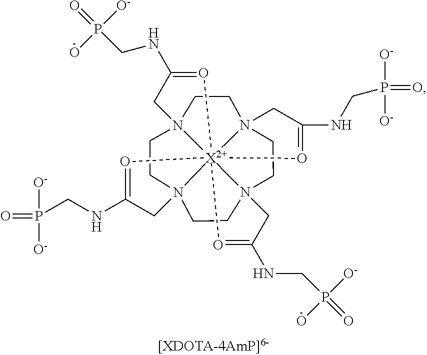

wherein X is selected from the group consisting of Fe, Zn, Co, Ni, Mn, Au, Pt, Cr, Pd, and Ti. In certain embodiments, the MRI/MRSI agent of Formula Ia is [XDOTA-4AmP]⁶⁻ wherein X is Fe. In other embodiments, the MRI/MRSI agent of Formula Ia is [XDOTA-4AmP]⁶⁻ wherein X is Co. In certain embodiments, the MRI/MRSI agent of Formula Ia is [XDOTA-4AmP]⁶⁻ wherein X is Ni. In certain embodiments, [XDOTA-4AmP]⁶⁻ exhibits high chemical shift sensitivity to temperature and pH. In certain embodiments, the exchangeable amide protons are sensitive to changes in pH and temperature. In certain embodiments, the phosphonate groups are sensitive to changes in pH.

In certain embodiments, the MRI/MRSI agent comprising a ligand of Formula II is an MRI/MRSI agent of Formula IIa:

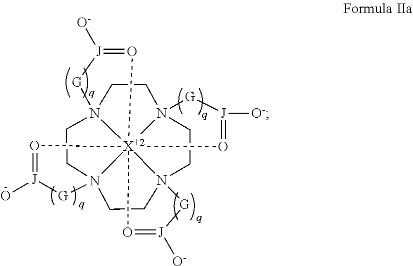

wherein:
each G is independently CR^{20a}R^{21a};
each J is independently C or P(O⁻);
each of R^{20a} and R^{21a} is independently selected from the group consisting of hydrogen, C₁-C₆ linear alkyl, and C₃-C₆ branched alkyl, wherein adjacent R^{20a} and R^{21b} groups may bond or fuse to form a C₃-C₁₂ cycloalkyl;
X is a transition metal selected from the group consisting of Fe, Zn, Co, and Ni, Mn, Au, Pt, Cr, Pd, and Ti; and
each q is independently an integer from 1-6.

In certain embodiments, each of J is C.

In certain embodiments, each J is P(O⁻).

In certain embodiments, each q is 1.

In certain embodiments, each of R^{20a} and R^{21a} is hydrogen.

In certain embodiments, at least one of R^{20a} and R^{21a} is a C₁-C₆ linear alkyl. In certain embodiments, each instance of R^{20a} is a C₁-C₆ linear alkyl. In certain embodiments, each instance of R^{20a} is methyl. In certain embodiments, each instance of R^{21a} is a C₁-C₆ linear alkyl.

In certain embodiments, each instance of R^{21a} is methyl.

In certain embodiments, X is Fe. In other embodiments, X is Co. In other embodiments, X is Ni.

In certain embodiments, the MRI/MRSI agent of Formula IIa is

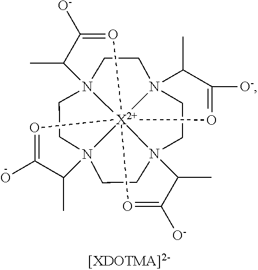

wherein X is selected from the group consisting of Fe, Zn, Co, Ni, Mn, Au, Pt, Cr, Pd, and Ti. In certain embodiments, the MRI/MRSI agent of Formula IIa is [XDOTMA]²⁻ wherein X is Fe. In other embodiments, the MRI/MRSI agent of Formula IIa is [XDOTMA]²⁻ wherein X is Co. In certain embodiments, the MRI/MRSI agent of Formula IIa is [XDOTMA]²⁻ wherein X is Ni. In certain embodiments, [XDOTMA]²⁻ exhibits high chemical shift sensitivity to temperature. In other embodiments, [XDOTMA]²⁻ exhibits high chemical shift sensitivity to pH. In other embodiments, [XDOTMA]²⁻ exhibits high chemical shift sensitivity to temperature and pH. In certain embodiments, the non-exchangeable protons of [XDOTMA]²⁻ are sensitive to changes in temperature. In certain embodiments, the non-exchangeable protons of the methyl groups of [XDOTMA]²⁻ are sensitive to changes in temperature. In other embodiments, the non exchangeable protons of the methyl groups of [XDOTMA]²⁻ are sensitive to changes in pH. In other embodiments, the non-exchangeable protons of the methyl groups of [XDOTMA]²⁻ are sensitive to changes in temperature and pH. In certain embodiments, the carboxylate groups are sensitive to changes in pH.

In another aspect, the present disclosure relates to a composition comprising an MRI/MRSI agent. In certain embodiments, the MRI/MRSI agent is an agent of Formula Ia. In other embodiments, the MRI/MRSI agent is an agent of Formula IIa. In certain embodiments, the MRI/MRSI agent is selected from the group consisting of [XDOTA-4AmC]²⁻, [XDOTA-4AmP]⁶⁻, [XDOTMA]²⁻, and combinations thereof, wherein X is selected from the group consisting of Fe, Co, and Ni. In certain embodiments, the composition comprises a solvent. The solvent can be any organic, biocompatible, or aqueous solvent known to a person of skill in the art. Exemplary organic solvents include, but are not limited to, methanol, ethanol, isopropanol, n-butanol, t-butanol, pentanes, hexanes, benzene, toluene, dichloromethane, chloroform, diethyl ether, dimethyl ether, ethyl acetate, dimethylformamide, and combinations thereof. Exemplary aqueous solvents include, but are not limited to, distilled water, deionized water, saline, Ringer's lactate solution, and combinations thereof. In certain embodiments, the composition comprises a biocompatible solvent. In certain embodiments, the biocompatible solvent is plasma. In certain embodiments, the plasma is pH neutral plasma.

In certain embodiments, the composition comprises one or more drugs or pharmaceutical agents. In certain embodiments, the drug or pharmaceutical agent is a drug or pharmaceutical agent used for the treatment of cancer (i.e. a chemotherapy drug or pharmaceutical agent). Exemplary chemotherapy drugs or pharmaceutical agents include, but are not limited to, cyclophosphamide, methotrexate, 5-fluorouracil, vinorelbine, doxorubicin, cyclophosphamide, docetaxel, bleomycin, vinblastine, dacarbazine, mustine, vincristine, procarbazine, prednisolone, etoposide, cisplatin, epirubicin, capecitabine, folinic acid, oxaliplatin, temozolomide, carmustine, bevacizumab, pembrolizumab, nivolumab, cemiplimab, atezolizumab, dostarlimab, durvalumab, avelumab, ipilimumab lomustine, everolimus, cetuximab, gefitinib, erlotinib, sorafenib, and combinations thereof. In some embodiments, the drug or pharmaceutical agent is a PD-1, PD-L1, or CTLA-4 checkpoint inhibitor. In certain embodiments, the composition comprises an inactive ingredient. The inactive ingredient may be any inactive ingredient known to a person of skill in the art. In certain embodiments, the inactive ingredient is selected from the group consisting of excipients, diluents, fillers, binders, disintegrants, lubricants, colorants, preservatives, surfactants, stabilizers, viscosity increasing agents, sweeteners, and any combinations thereof. In certain embodiments, the inactive ingredient is a pharmaceutically acceptable carrier. Exemplary pharmaceutical carriers are described elsewhere herein.

### Methods

In another aspect, the present disclosure relates to a method of monitoring tissue temperature and/or pH in a patient in need thereof, the method comprising: administering to the patient an MRI/MRSI agent of Formula Ia or Formula IIa; and measuring a change in the chemical shift of one or more protons in the MRI/MRSI agent.

In certain embodiments, the MRI/MRSI agent is an agent of Formula Ia. In certain embodiments, the agent of Formula Ia is [XDOTA-4AmC]²⁻ or [XDOTA-4AmP]⁶⁻, wherein X is selected from the group consisting of Fe, Zn, Co, Ni, Mn, Au, Pt, Cr, Pd, and Ti. In certain embodiments, the agent of Formula Ia is [XDOTA-4AmC]²⁻ or [XDOTA-4AmP]⁶⁻, wherein X is selected from the group consisting of Fe, Co, and Ni. In other embodiments, the MRI/MRSI agent is an agent of Formula IIa. In certain embodiments, the agent of Formula IIa is [XDOTMA]²⁻, wherein X is selected from the group consisting of Fe, Zn, Co, Ni, Mn, Au, Pt, Cr, Pd, and Ti. In certain embodiments, the agent of Formula IIa is [XDOTMA]²⁻, wherein X is selected from the group consisting of Fe, Co, and Ni.

The MRI/MRSI agent can be administered to a patient using any technique known to a person of skill in the art. Exemplary administration methods include, but are not limited to, oral administration, intravenous administration, subcutaneous administration, intramuscular administration, intra-articular administration, and topical administration. In certain embodiments, the MRI/MRSI agent is administered to the patient as a component of a composition. In certain embodiments, the composition comprises an aqueous solvent. Exemplary aqueous solvents are described elsewhere herein.

In certain embodiments, the MRI/MSRI agent is administered to the patient at the site of a tumor. In certain embodiments, the MRI/MSRI agent is used to monitor the pH and/or temperature of the tumor microenvironment. In certain embodiments, the transition metal ion in the MRI/MSRI agent acts as a contrast agent. The MRI/MRSI agent can be administered to a patient using any dosage known to person of skill in the art of MRI/MRSI agents. In certain embodiments, the maximum dosage of MRI/MRSI agent is between about 0.1 mmol/kg and 100 mmol/kg. In certain embodiments, the maximum dosage of MRI/MRSI agent is between about 0.1 mmol/kg and 90 mmol/kg. In certain embodiments, the maximum dosage of MRI/MRSI agent is between about 0.1 mmol/kg and 80 mmol/kg. In certain embodiments, the maximum dosage of MRI/MRSI agent is between about 0.1 mmol/kg and 70 mmol/kg. In certain embodiments, the maximum dosage of MRI/MRSI agent is between about 0.1 mmol/kg and 60 mmol/kg. In certain embodiments, the maximum dosage of MRI/MRSI agent is between about 0.1 mmol/kg and 50 mmol/kg. In certain embodiments, the maximum dosage of MRI/MRSI agent is between about 0.1 mmol/kg and 40 mmol/kg. In certain embodiments, the maximum dosage of MRI/MRSI agent is between about 0.1 mmol/kg and 30 mmol/kg. In certain embodiments, the maximum dosage of MRI/MRSI agent is between about 0.1 mmol/kg and 20 mmol/kg. In certain embodiments, the maximum dosage of MRI/MRSI agent is between about 0.1 mmol/kg and 10 mmol/kg. In certain embodiments, the maximum dosage of MRI/MRSI agent is between about 0.5 mmol/kg and 5 mmol/kg.

In some embodiments, the step of administering to the patient an MRI/MRSI agent is preceded by the step of administering to the patient a drug or pharmaceutical. In certain embodiments, the drug or pharmaceutical is a chemotherapy drug or pharmaceutical. Exemplary chemotherapy drugs or pharmaceuticals are described elsewhere herein. In certain embodiments, the chemotherapy drug or pharmaceutical is a drug or pharmaceutical used to treat glioblastoma. In certain embodiments, the drug or pharmaceutical is used to treat a malignant glioma. In certain embodiments, the drug or pharmaceutical is used to treat glioblastoma multiforme (GBM).

In certain embodiments, the step of measuring the change in the chemical shift of one or more protons in the MRI/MRSI agent comprises measuring the chemical shift of a proton that is sensitive to changes in pH, temperature, or both pH and temperature. In certain embodiments, the exchangeable and non-exchangeable protons on the polyazamacrocyclic ligand of the MRI/MRSI agent are sensitive to changes in pH, temperature, or both pH and temperature. In certain embodiments, the MRI/MRSI agent is [XDOTA-4AmC]²⁻ wherein X is any transition metal described elsewhere herein, and wherein the —NH protons are sensitive to changes in pH, temperature, or pH and temperature. In certain embodiments, the MRI/MRSI agent is [XDOTA-4AmC]²⁻, wherein X is selected from Fe, Co, and Ni, and wherein the —NH protons are sensitive to changes in pH, temperature, or pH and temperature. In other embodiments, the MRI/MRSI agent is [XDOTA-4AmP]⁶⁻, wherein X is any transition metal described elsewhere herein, and wherein the —NH protons are sensitive to changes in pH, temperature, or pH and temperature. In other embodiments, the MRI/MRSI agent is [XDOTA-4AmP]⁶⁻, wherein X is selected from Fe, Co, and Ni, and wherein the —NH protons are sensitive to changes in pH, temperature, or pH and temperature. In other embodiments, the MRI/MRSI agent is [XDOTMA]² wherein X is any transition metal described elsewhere herein, and wherein the —CH₃ protons are sensitive to changes in pH, temperature, or pH and temperature. In other embodiments, the MRI/MRSI agent is [XDOTA-4AmP]⁶⁻, wherein X is selected from Fe, Co, and Ni, and wherein the —CH₃ protons are sensitive to changes in pH, temperature, or pH and temperature. Although not wishing to be limited by theory, it is believed that changes in temperature and/or pH result in structural variations of the MRI/MRSI agent, which in turn induces chemical shift variations. In certain embodiments, the chemical shift variations are measured using the BIRDS (Biosensor Imaging of Redundant Deviation Shifts) method.

In certain embodiments, the change in the chemical shift of one or more protons in the MRI/MRSI agent is used to monitor the efficacy of cancer treatment in a patient in need thereof. In certain embodiments, the change in the chemical shift of one or more protons in the MRI/MRSI agent is used to monitor tumor growth in a patient in need thereof. In certain embodiments, the change in the chemical shift of one or more protons in the MRI/MRSI agent is used to map extracellular pH (pHe) inside and outside of a tumor (i.e. the intratumoral-peritumoral pHe gradient) in a patient in need thereof. In certain embodiments, the change in the chemical shift of one or more protons in the MRI/MRSI agent is used to map temperature both inside and outside of a tumor in a patient in need thereof.

In yet another aspect, the present disclosure relates to a method of monitoring the efficacy of a cancer treatment in a patient in need thereof. In certain embodiments, the method comprises administering to the patient a chemotherapy drug or pharmaceutical; administering to the patient, at the site of a tumor, an MRI/MRSI agent of Formula Ia or Formula IIa; and measuring a change in the chemical shift of one or more protons in the MRI/MRSI agent.

The chemotherapy drug or pharmaceutical agent may be any chemotherapy drug or pharmaceutical known to a person of skill in the art. Exemplary chemotherapy drugs or pharmaceuticals are described elsewhere herein. The chemotherapy drug or pharmaceutical can be administered using any technique known to a person of skill in the art. Exemplary administration methods are described elsewhere herein. In certain embodiments, the chemotherapy drug or pharmaceutical is a drug or pharmaceutical used to treat glioblastoma. In certain embodiments, the drug or pharmaceutical is used to treat a malignant glioma. In certain embodiments, the drug or pharmaceutical is used to treat glioblastoma multiforme (GBM). In certain embodiments, the chemotherapy drug or pharmaceutical is temozolomide, sorafenib, or a combination thereof.

In certain embodiments, the MRI/MRSI agent is an agent of Formula Ia. In certain embodiments, the agent of Formula Ia is [XDOTA-4AmC]²⁻ or [XDOTA-4AmP]⁶⁻, wherein X is selected from the group consisting of Fe, Zn, Co, Ni, Mn, Au, Pt, Cr, Pd, and Ti. In certain embodiments, the agent of Formula Ia is [XDOTA-4AmC]²⁻ or [XDOTA-4AmP]⁶⁻, wherein X is selected from the group consisting of Fe, Co, and Ni. In other embodiments, the MRI/MRSI agent is an agent of Formula IIa. In certain embodiments, the agent of Formula IIa is [XDOTMA]²⁻, wherein X is selected from the group consisting of Fe, Zn, Co, Ni, Mn, Au, Pt, Cr, Pd, and Ti. In certain embodiments, the agent of Formula IIa is [XDOTMA]²⁻, wherein X is selected from the group consisting of Fe, Co, and Ni. The MRI/MRSI agent can be administered to a patient using any technique known to a person of skill in the art. Exemplary administration methods are described elsewhere herein. The MRI/MRSI agent can be administered to a patient at any dosage known to a person of skill in the art of MRI/MRSI agents. Exemplary maximum dosages of the MRI/MRSI agent are described elsewhere herein.

In certain embodiments, the MRI/MRSI agent is administered to the patient as a component of a composition. In certain embodiments, the composition comprises an aqueous solvent. Exemplary aqueous solvents are described elsewhere herein. In certain embodiments, a composition comprising the MRI/MRSI agent is injected into a patient's tissue at the site of a tumor. In certain embodiments, the MRI/MSRI agent is used to monitor the pH, temperature, or pH and temperature of the tumor microenvironment. In certain embodiments, the transition metal ion in the MRI/MSRI agent acts as a contrast agent.

In certain embodiments, the step of measuring the change in the chemical shift of one or more protons in the MRI/MRSI agent comprises measuring the chemical shift of a proton that is sensitive to changes in pH and/or temperature. In certain embodiments, the exchangeable and non-exchangeable protons on the polyazamacrocyclic ligand of the MRI/MRSI agent are sensitive to changes in pH, temperature, or both pH and temperature. In certain embodiments, the MRI/MRSI agent is [XDOTA-4AmC]²⁻, wherein X is any transition metal described elsewhere herein, and wherein the —NH protons are sensitive to changes in pH, temperature, or pH and temperature. In certain embodiments, the MRI/MRSI agent is [XDOTA-4AmC]²⁻, wherein X is selected from Fe, Co, and Ni, and wherein the —NH protons are sensitive to changes in pH, temperature, or pH and temperature. In other embodiments, the MRI/MRSI agent is [XDOTA-4AmP]⁶⁻, wherein X is any transition metal described elsewhere herein, and wherein the —NH protons are sensitive to changes in pH, temperature, or pH and temperature. In other embodiments, the MRI/MRSI agent is [XDOTA-4AmP]⁶⁻, wherein X is selected from Fe, Co, and Ni, and wherein the —NH protons are sensitive to changes in pH, temperature, or pH and temperature. In other embodiments, the MRI/MRSI agent is [XDOTMA]²⁻, wherein X is any transition metal described elsewhere herein, and wherein the —CH₃ protons are sensitive to changes in pH, temperature, or pH and temperature. In other embodiments, the MRI/MRSI agent is [XDOTA-4AmP]⁶⁻, wherein X is selected from Fe, Co, and Ni, and wherein the —CH₃ protons are sensitive to changes in pH, temperature, or pH and temperature. Although not wishing to be limited by theory, it is believed that changes in temperature and/or pH result in structural variations of the MRI/MRSI agent, which in turn induces chemical shift variations. In certain embodiments, the chemical shift variations are measured using the BIRDS (Biosensor Imaging of Redundant Deviation Shifts) method.

In some embodiments, the step of measuring a change in the chemical shift of one or more protons in the MRI/MRSI agent further comprises the step of mapping the extracellular pH inside and outside of the tumor. In some embodiments, the step of measuring a change in the chemical shift of one or more protons in the MRI/MRSI agent further comprises the step of mapping the temperature inside and outside of the tumor.

### EXPERIMENTAL EXAMPLES

The disclosure is further described in detail by reference to the following experimental examples. These examples are provided for purposes of illustration only, and are not intended to be limiting unless so specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples therefore, specifically point out the preferred embodiments of the present disclosure, and are not to be construed as limiting in any way the remainder of the disclosure.

### Example 1: Transition Metal Macrocyclic Complexes MRI Contrast Agents for Molecular Imaging

### Materials and Methods

### DOTA-Based Ligands

### Ligand 1: [DOTA-4AmC]⁴⁻

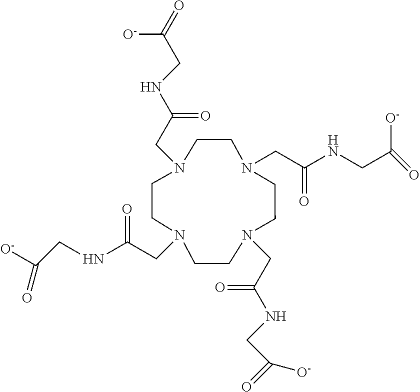 [SMILES: O=C([O-])CNC(=O)CN1CCN(CC(=O)NCC(=O)[O-])CCN(CC(=O)NCC(=O)[O-])CCN(CC(=O)NCC(=O)[O-])CC1]

### 1,4,7,10-tetraazacyclododecane-1,4,7,10-tetrakis(acetamidoacetate)

### Ligand 2: [DOTA-4AmP]⁸⁻

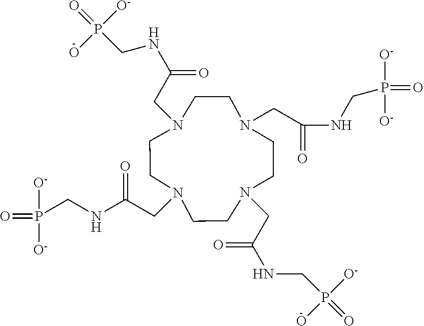 [SMILES: O=C(CN1CCN(CC(=O)NCP(=O)([O-])O)CCN(CC(=O)NCP(=O)([O-])[O-])CCN(CC(=O)NCP(=O)([O-])O)CC1)NCP(=O)([O-])[O-]]

### 1,4,7,10-tetraazacyclododecane-1,4,7,10-tetrakis(acetamido-methylenephosphonate)

### Ligand 3: [DOTMA]⁴⁻

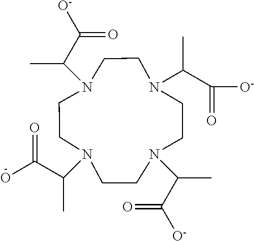 [SMILES: CC(C(=O)[O-])N1CCN(C(C)C(=O)[O-])CCN(C(C)C(=O)O)CCN(C(C)C(=O)[O-])CC1]

(1R,4R,7R,10R)-α,α′,α″,α′″-tetramethyl-1,4,7,10-tetraazacyclododecane-1,4,7,10-tetraacetate

### Synthesis of Complexes

The [XDOTA-4AmC]²⁻, [XDOTA-4AmP]⁶⁻, and [XDOTMA]²⁻ complexes of the disclosure are prepared by refluxing a [XDOTA-4AmC]⁴⁻, [XDOTA-4AmP]⁸⁻, or [XDOTMA]⁴⁻ ligand with a transition metal salt comprising a paramagnetic transition metal X, wherein X includes but is not limited to, iron, zinc, cobalt, nickel, copper, manganese, gold, platinum, chromium, palladium, or titanium.

The example below shows a synthesis of a [XDOTA-4AmC]²⁻ complex comprising iron, cobalt, or nickel.

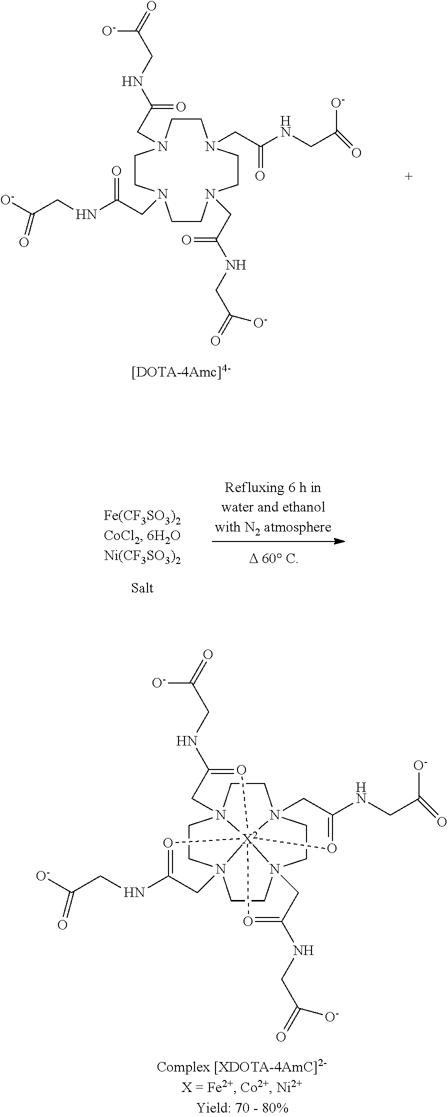

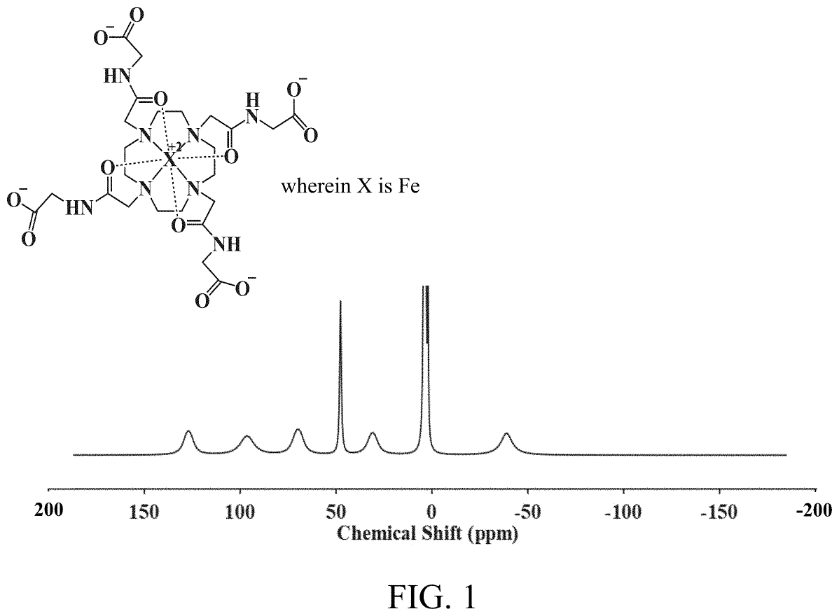

The ¹H NMR spectra of [FeDOTA-4Amc]²⁻ is depicted in FIG. 1.

The example below shows a synthesis of a [XDOTA-4AmP]²⁻ complex comprising iron, cobalt, or nickel.

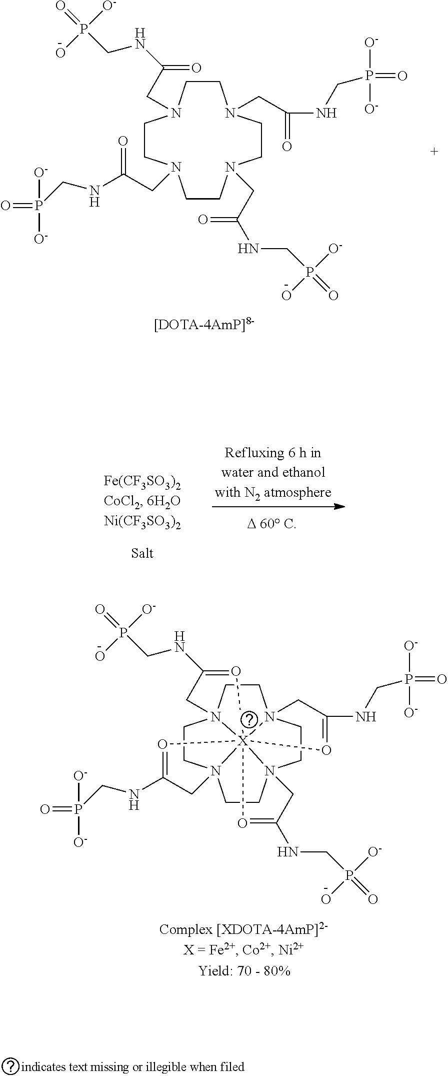

The example below shows a synthesis of a [XDOTMA]²⁻ complex comprising iron, cobalt, or nickel.

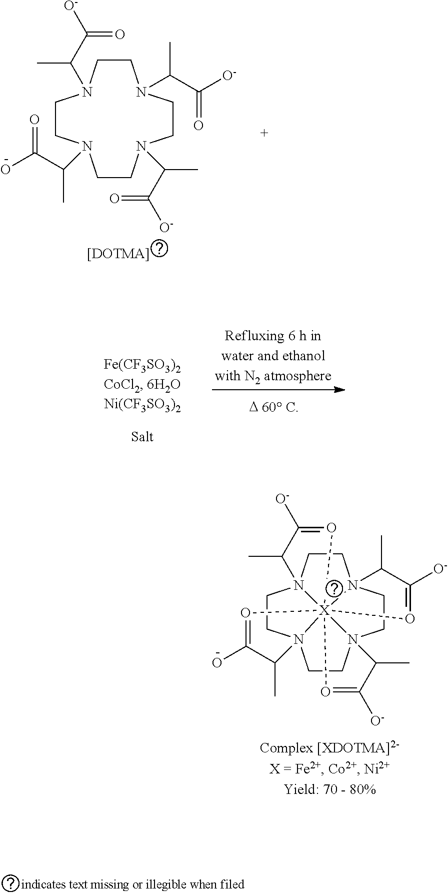

The recently developed BIRDS method overcomes the limitations of common MRI/MRSI methods including the low sensitivity of ³¹P-MRSI methods, the instability of fluorinated compounds and the installation of fluorine coils required for the ¹⁹F-MRSI methods, and the high concentrations needed for CEST methods, by instead using the chemical shifts of non-exchangeable protons from paramagnetic complexes. Since temperature and pH changes result in structural variations of the chelates, inducing also chemical shift variations, BIRDS utilizes these chemical shifts for molecular reporting to provide simultaneous measurements of temperature and pH. A prerequisite for a probe to be a good BIRDS agent is to have the appropriate functional group, (e.g. a phosphonate group for pH sensing).

Most of the MRI or MRSI methods for measuring temperature and pH utilize exogenous agents composed of paramagnetic lanthanide ions (e.g., Gd³⁺, Eu³⁺, Yb³⁺, Tm³⁺) complexes from derivatives of CYCLEN-based ligand 1,4,7,10-tetraazacyclododecane-1,4,7,10-tetraacetic acid or DOTA, such as, 1,4,7,10-tetraazacyclododecane-1,4,7,10-tetrakis(acetamidoacetate) [DOTA-4AmC]⁴⁻, 1,4,7,10-tetraazacyclododecane-1,4,7,10-tetrakis(acetamido-methylenephosphonate) [DOTA-4AmP]⁸⁻ and (1R,4R,7R,10R)-α,α′,α″,α′″-tetramethyl-1,4,7,10-tetraazacyclododecane-1,4,7,10-tetraacetate [DOTMA]⁴⁻. All of these ligands have non-exchangeable protons which are paramagnetically shifted due to their close proximity to the Ln³⁺ ion. The phosphonate group in [DOTA-4AmP]⁸⁻ and the carboxylate group in [DOTA-4AmC]⁴⁻ and [DOTMA]⁴⁻ on the pendent arms are responsible for pH sensing. In addition, there are exchangeable amide (—NH) protons in [DOTA-4AmC]⁴⁻ and [DOTA-4AmP]⁸⁻ that are responsible for PARACEST contrast.

Contrast agents based on endogenous first row transition metal (Fe²⁺, Co²⁺ or Ni²⁺) complexes can provide a more bio-compatible alternative to the lanthanide ions (e.g. Gd³⁺, Eu³⁺, Yb³⁺, Tm³⁺), because they are better tolerated by animals or humans. The coordinating ligand for lanthanide ions is mostly CYCLEN-based, whereas the favorable ligand for transition ions seems to be 1,4,7-triazacyclononane (TACN). TACN provides one less pendant arm for electron donating compared to CYCLEN.

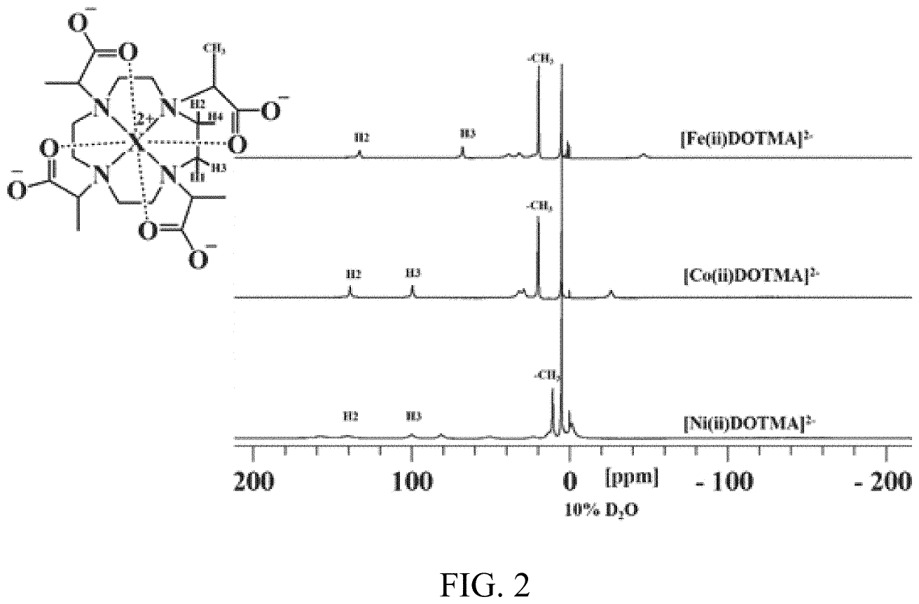

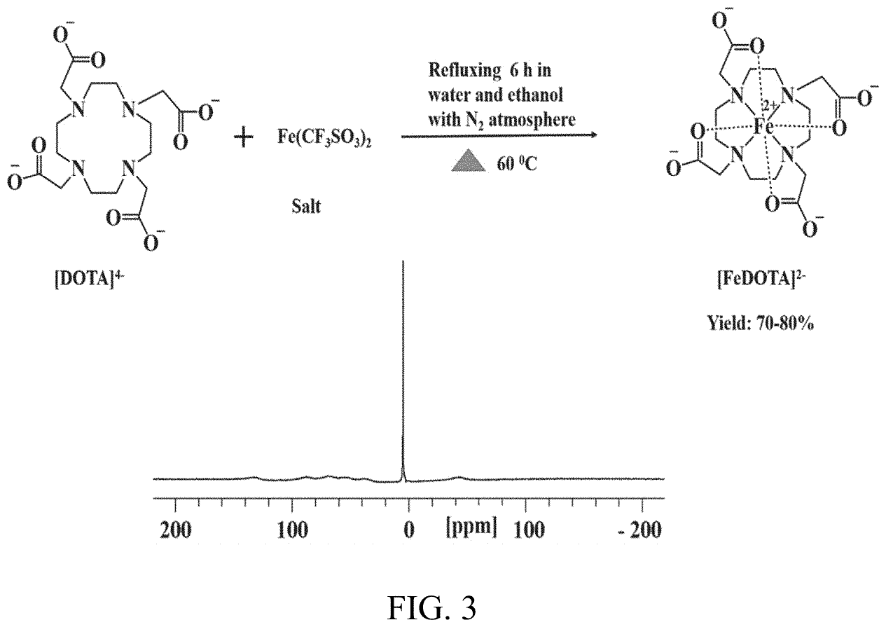

The present disclosure relates, in part, to BIRDS agents containing transition metal ions as paramagnetic center complexed with ligands having exchangeable and nonexchangeable protons in order to provide simultaneous temperature and pH measurements. The present disclosure is based, in part, on the discovery that the ligands [DOTA-4AmC]⁴⁻ and [DOTA-4AmP]⁸⁻ complexed with transition metal ions (Fe²⁺, Co²⁺ or Ni²⁺) show that the —NH (amide) proton have high temperature and pH sensitivities, which can be used for temperature and pH detection. Stable complexes of [XDOTMA]²⁻ (where X is Fe²⁺, Co²⁺ or Ni²⁺) show several nonexchangeable proton signals (H2, H3 and —CH₃) (FIG. 2) with high temperature sensitivity, whereas the analogous complex of [FeDOTA]²⁻ show only extremely broad proton signals (FIG. 3). The presently disclosed contrast agents have certain non-limiting advantages. In one non-limiting aspect, they are more bio-compatible when compared to the existing lanthanide ions-based contrast agents. In another non-limiting aspect, they support the BIRDS method, which is inherently independent of agent concentration and static magnetic field. In yet another non-limiting aspect, with regard to BIRDS agents [XDOTA-4AmC]²⁻ and [XDOTA-4AmP]⁶⁻ where X is Fe²⁺, Co²⁺ or Ni²⁺, the —NH proton chemical shift varies as a function of temperature and pH. Therefore, these agents have advantages over the amide-based CEST agents where higher concentration is required.

For paramagnetic complexes, the total chemical shift of the nuclear spin (δ_{O}) is dependent on the cumulative effects of diamagnetic (δ_{D}) and paramagnetic (δ_{P}) terms, where δ_{P} is typically much larger than δ_{D}, which has also a very weak temperature dependence. Thus, the effect of temperature on the chemical shift is dominated by δ_{P}. Similarly, protonation of an exchangeable group from the complex can modify its geometry and alter its chemical shifts, thus providing pH sensing capability. Variation of the total shift term, Δδ_{O}, when temperature changes by ΔT and pH changes by ΔpH, can be modeled as

Δδ_{O}=C_{T}ΔT+C_{pH}ΔpH+C_{X}Δ[X]

where C_{T}=(Δδ_{O}/ΔT)_{pH,[X]} is the temperature sensitivity, C_{pH}=(Δδ_{O}/Δ pH)_{T,[X]} is the pH sensitivity, and C_{X} represents the sensitivity to cation X concentration (see Coman, D. et al., “Brain temperature by Biosensor Imaging of Redundant Deviation in Shifts (BIRDS): comparison between TmDOTP5⁻ and TmDOTMA⁻,” NMR in Biomedicine, 2010, 23(3):277-285 for details). pH or temperature sensing with BIRDS depends on C_{T} and C_{pH} in relation to Δδ_{O} (>>10¹ ppm) and is inherently independent of agent concentration and static magnetic field (B_{O}) (Coman, D. et al., “Brain temperature and pH measured by 1H chemical shift imaging of a thulium agent,” NMR in Biomedicine, 2009, 22(2):229-239).

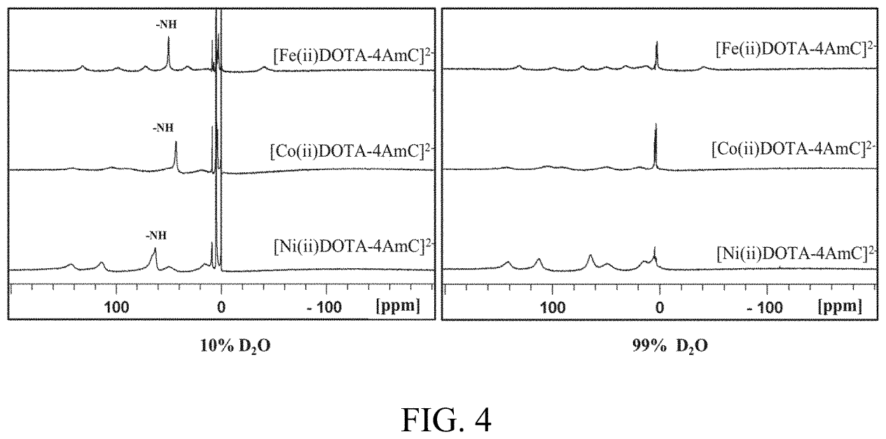

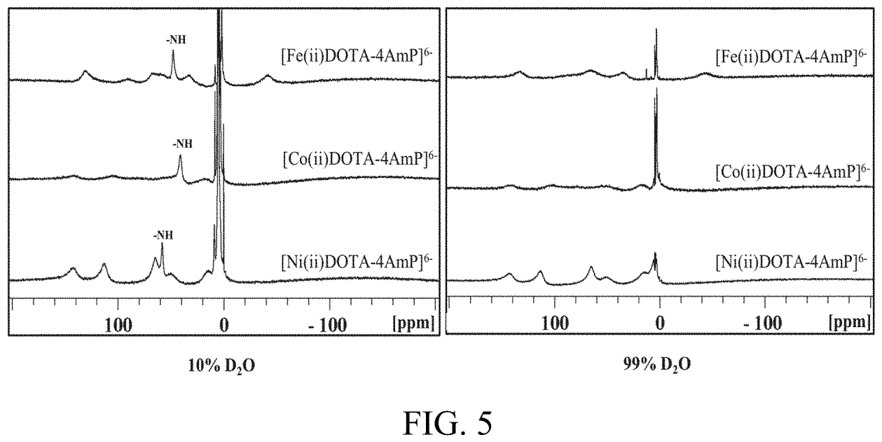

The —NH groups of the pendent arms in complexes [XDOTA-4AmC]²⁻ and [XDOTA-4AmP]⁶⁻ are chemically equivalent and close to the paramagnetic metal ion center. As a result, the ¹H NMR signal of the —NH proton is paramagnetically well-shifted from the bulk water signal. The ¹H NMR signal of —NH proton decreases upon addition of deuterium oxide (D₂O˜10% of sample volume) providing the evidence of chemical exchange with bulk water protons. An amide-based contrast agent mixed with ˜10% D₂O does not show any —NH proton peak in its ¹H NMR spectra. The ¹H NMR signal of the amide proton in [XDOTA-4AmC]²⁻ and [XDOTA-4AmP]⁶⁻ complexes decreases in 10% D₂O and almost disappears in 99% D₂O (see FIG. 4 and FIG. 5, respectively). Furthermore, none of the two complexes show any CEST signals. All of this evidence clearly demonstrates that the exchange rate of the amide proton in these complexes is slower than the protons of conventional paramagnetic CEST agent. Another important feature of these complexes, the ¹H NMR signal of amide proton is the highest peak among all protons in the corresponding molecule.

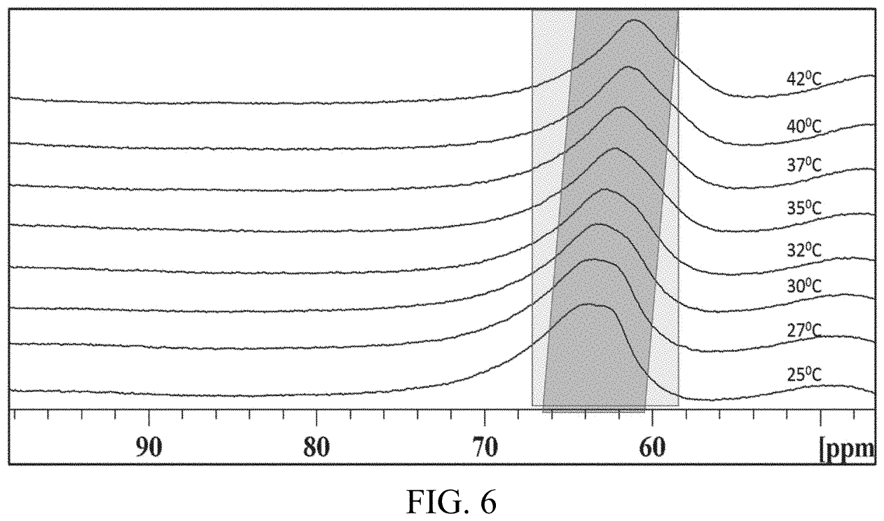

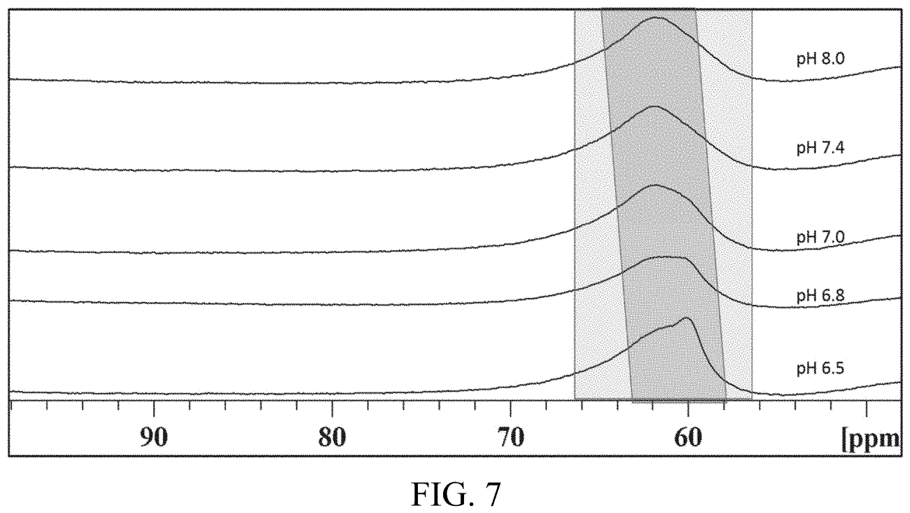

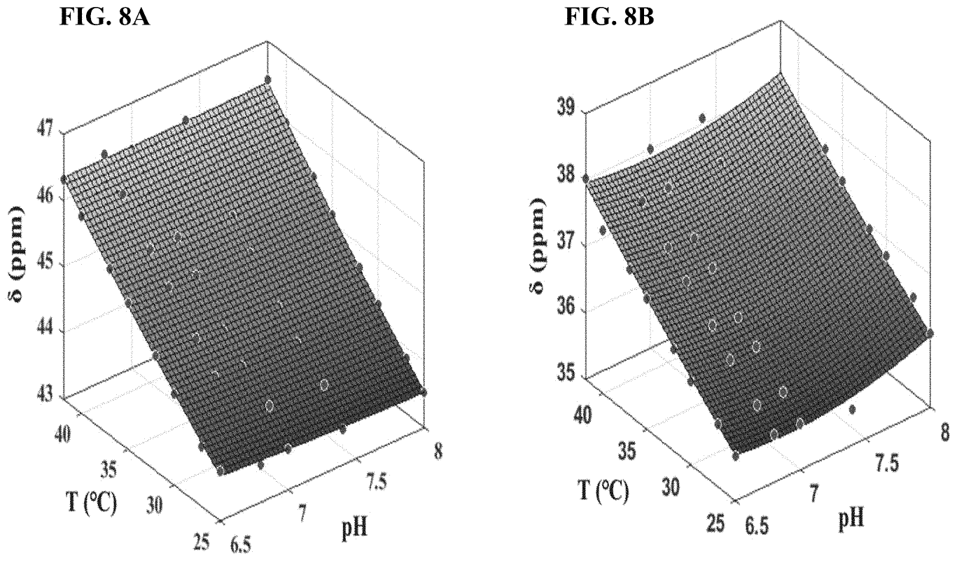

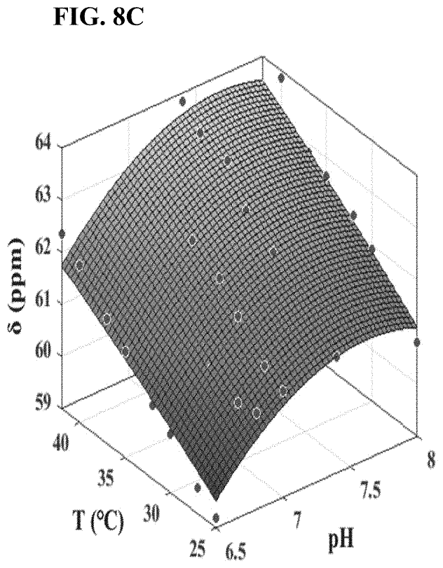

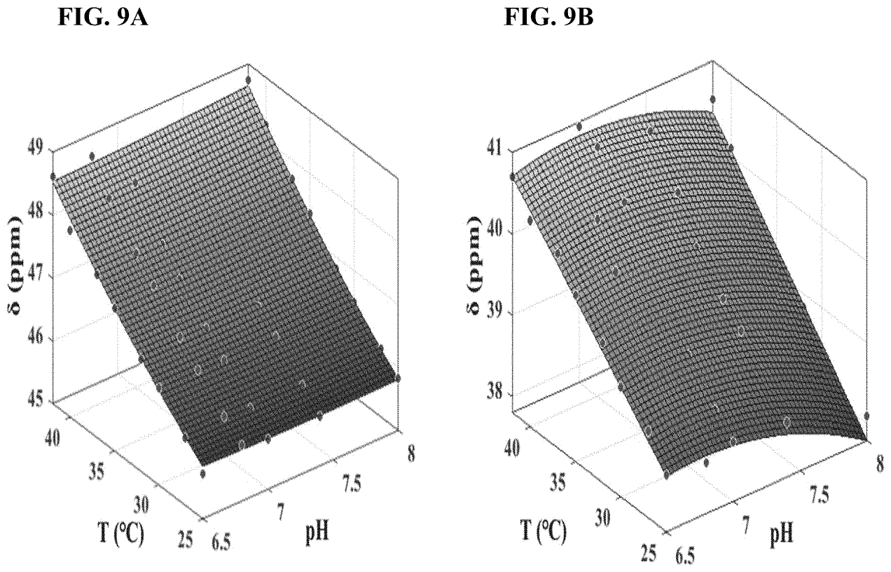

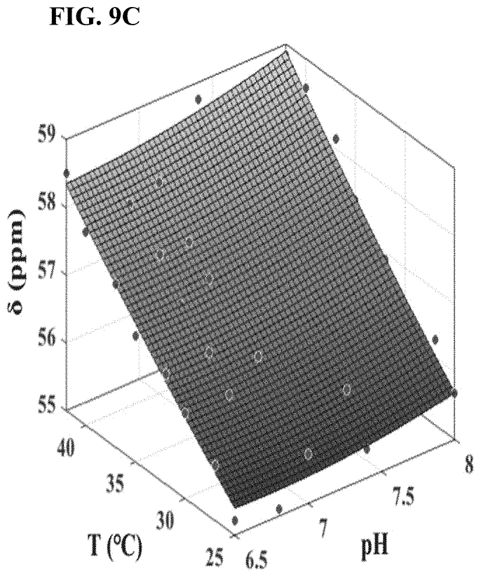

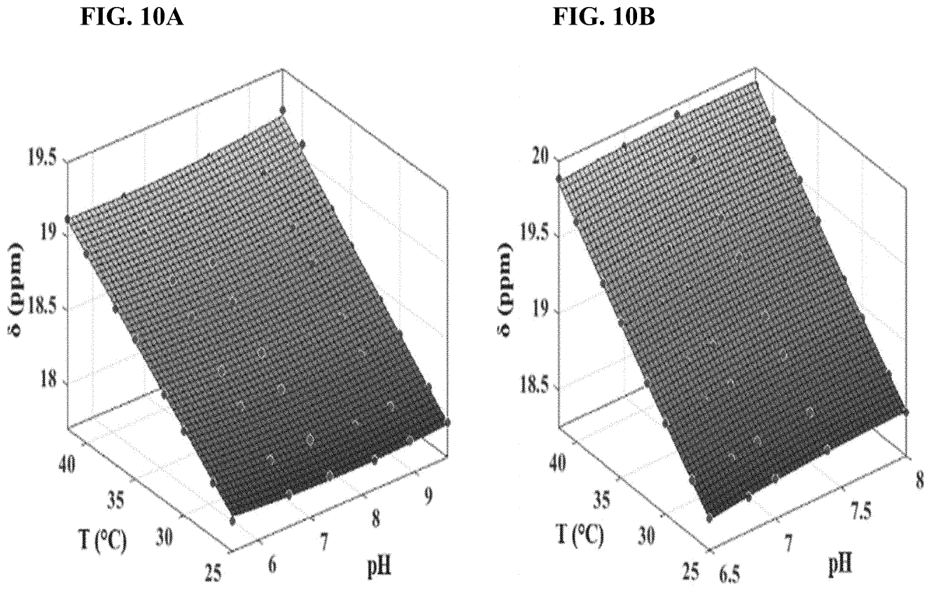

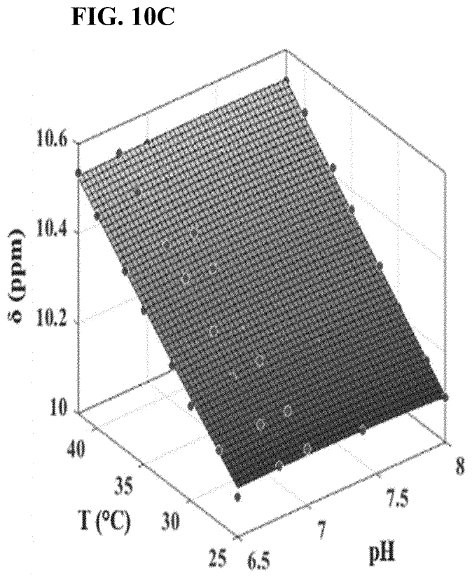

Since the ¹H NMR chemical shifts of the —NH peaks in the [XDOTA-4AmC]²⁻ and [XDOTA-4AmP]⁶⁻ complexes are temperature and pH-dependent (FIG. 6 and FIG. 7), they can be used for measurements of temperature and pH using BIRDS (Table 1). The 3D surface plots of [XDOTA-4AmC]²⁻ and [XDOTA-4AmP]⁶⁻ complexes in FIGS. 8A-8C and FIGS. 9A-9C, respectively, demonstrate the dependence of the —NH proton chemical shift, δ, as a function of temperature T and pH. In the [XDOTMA]²⁻ set of complexes, it was observed that the —CH₃ proton resonance has a higher intensity compared to other nonexchangeable proton peaks, like H₂ or H₃ (FIG. 2). The 3D surface plots in FIGS. 10A-C represent the temperature and pH dependencies of the —CH₃ proton resonance of the [XDOTMA]²⁻ complex.

TABLE 1
Temperature and pH sensitivities of proton
resonances in complexes of the disclosure.

| | | Temperature | |
|---|---|---|---|
| | Proton | Sensitivity | pH Sensitivity |
| Complex | Resonance | (ppm/° C.)^{a} | (ppm/pH unit)^{b} |
| [Fe(ii)DOTA-4AmC]²⁻ | —NH | 0.16 ± 0.01 | 0.04 ± 0.02 |
| [Co(ii)DOTA-4AmC]²⁻ | —NH | 0.13 ± 0.01 | 0.25 ± 0.04 |
| [Ni(ii)DOTA-4AmC]²⁻ | —NH | 0.14 ± 0.02 | 1.07 ± 0.12 |
| [Fe(ii)DOTA-4AmP]⁶⁻ | —NH | 0.16 ± 0.01 | 0.06 ± 0.02 |
| [Co(ii)DOTA-4AmP]⁶⁻ | —NH | 0.14 ± 0.01 | 0.37 ± 0.08 |
| [Ni(ii)DOTA-4AmP]⁶⁻ | —NH | 0.18 ± 0.01 | 0.29 ± 0.09 |
| [Fe(ii)DOTMA]²⁻ | —CH₃ | 0.072 ± 0.001 | 0.009 ± 0.004 |
| [Co(ii)DOTMA]²⁻ | —CH₃ | 0.082 ± 0.001 | 0.045 ± 0.007 |
| [Ni(ii)DOTMA]²⁻ | —CH₃ | 0.025 ± 0.001 | 0.005 ± 0.003 |

^{a}At 11.7 T and a temperature range of 25-42° C.
^{b}At 11.7 T and a pH range of 6.5-8.0

In various embodiments, compounds of Formula Ia and/or IIa or compositions containing compounds of Formula Ia and/or IIa have temperature sensitivities of about 0.01 to about 0.20, about 0.01 to about 0.10, or about 0.10 to about 0.20 ppm/° C. when measured at a magnetic field strength of 11.7 T. Compounds of Formula Ia and/or IIa or compositions containing compounds of Formula Ia and/or IIa have temperature sensitivities of at least about, less than about, or equal to about 0.01, 0.02, 0.03, 0.04, 0.05, 0.06, 0.07, 0.08. 0.09, 0.10, 0.11, 0.12, 0.13, 0.14, 0.15, 0.16, 0.17, 0.18, 0.19, or about 0.20 ppm/° C. when measured at a magnetic field strength of 11.7 T.

In various embodiments, compounds of Formula Ia and/or IIa or compositions containing compounds of Formula Ia and/or IIa can have pH sensitivities of about 0.001 to about 1.5, 0.001 to about 0.2, or about 0.1 to about 1.5 ppm/pH unit when measured in a magnetic field of 11.7 T and between a pH range of about 6.5 to about 8. Compounds of Formula Ia and/or IIa or compositions containing compounds of Formula Ia and/or IIa can have pH sensitivities of at least about, less than about, or equal to about 0.001, 0.002, 0.004, 0.006, 0.008, 0.01, 0.012, 0.014, 0.016, 0.018, 0.02, 0.022, 0.024, 0.026, 0.028, 0.03, 0.032, 0.034, 0.036, 0.038, 0.04, 0.042, 0.044, 0.046, 0.048, 0.05, 0.052, 0.054, 0.056, 0.058, 0.06, 0.062, 0.064, 0.066, 0.068, 0.07, 0.072, 0.074, 0.076, 0.078, 0.08, 0.082, 0.084, 0.086, 0.088, 0.09, 0.092, 0.094, 0.096, 0.098, 0.1, 0.102, 0.104, 0.106, 0.108, 0.11, 0.112, 0.114, 0.116, 0.118, 0.12, 0.122, 0.124, 0.126, 0.128, 0.13, 0.132, 0.134, 0.136, 0.138, 0.14, 0.142, 0.144, 0.146, 0.148, 0.15, 0.152, 0.154, 0.156, 0.158, 0.16, 0.162, 0.164, 0.166, 0.168, 0.17, 0.172, 0.174, 0.176, 0.178, 0.18, 0.182, 0.184, 0.186, 0.188, 0.19, 0.192, 0.194, 0.196, 0.198, or about 0.2 ppm/pH unit when measured in a magnetic field of 11.7 T and between a pH range of about 6.5 to about 8.

Compounds of Formula Ia and/or IIa or compositions containing compounds of Formula Ia and/or Ha can have pH sensitivities of at least about, less than about, or equal to about 0.01, 0.02, 0.04, 0.06, 0.08, 0.1, 0.12, 0.14, 0.16, 0.18, 0.2, 0.22, 0.24, 0.26, 0.28, 0.3, 0.32, 0.34, 0.36, 0.38, 0.4, 0.42, 0.44, 0.46, 0.48, 0.5, 0.52, 0.54, 0.56, 0.58, 0.6, 0.62, 0.64, 0.66, 0.68, 0.7, 0.72, 0.74, 0.76, 0.78, 0.8, 0.82, 0.84, 0.86, 0.88, 0.9, 0.92, 0.94, 0.96, 0.98, 1, 1.02, 1.04, 1.06, 1.08, 1.1, 1.12, 1.14, 1.16, 1.18, 1.2, 1.22, 1.24, 1.26, 1.28, 1.3, 1.32, 1.34, 1.36, 1.38, 1.4, 1.42, 1.44, 1.46, 1.48, or about 1.5 ppm/pH unit when measured in a magnetic field of 11.7 T and between a pH range of about 6.5 to about 8.

The contrast agents of the disclosure can be used to map a patient's pHe, therefore providing a therapeutic readout of drug delivery. For example, the contrast agents of the disclosure can be used to map drug delivery in cancers such as, but not limited to, glioblastoma. The prognosis remains dismal for most brain tumor patients. Malignant gliomas, including glioblastoma multiforme (GBM), fail treatments because gliomas invade outside tumor boundaries conventionally demarked by MRI contrast and the blood-brain barrier (BBB) blocks most drugs. To meet the need for MR readouts of the tumor physicochemical state, the BIRDS agents of the disclosure were developed to map the intratumoral-peritumoral pHe gradient and it was found that these agents provide a sensitive readout of cancer growth and treatment. Based on preliminary data obtained from GBM models (e.g. U251), including patient-derived xenograft (PDX) models, high-resolution pHe mapping with BIRDS will be validated as a therapeutic readout of chemotherapy drugs delivered into human GBM models. Although 1-2 mm diameter tumors were detected with BIRDS using non-methylated agents, higher resolution mapping of intratumoral-peritumoral pHe gradients can be reached with methylated contrast agents such as the [XDOTMA]²⁻ complexes of the disclosure. The intratumoral-peritumoral pHe gradient mapping by BIRDS can be validated using fluorescent pHe probes wherein the change in intratumoral-peritumoral pHe gradients with tumor aggression can be monitored. The compatibility of pHe mapping with BIRDS for tracking response to chemotherapy drugs (e.g. Temozolomide and Sorafenib) used to treat GBMs can also be tested. Both of these drugs are known to cross the BBB and are used in GMB therapy. Temozolomide activates apoptosis by alkylating DNA to stall cell replication and Sorafenib is a multiple kinase inhibitor targeting several oncogenic pathways and enhances glycolysis. In certain embodiments, pHe mapping by BIRDS enable monitoring of therapeutic response of various chemotherapy drugs for preclinical PDX models to potentially be translated clinically.

### ENUMERATED EMBODIMENTS

The following enumerated embodiments are provided, the numbering of which is not to be construed as designating levels of importance.

Embodiment 1 provides a compound of Formula Ia or Formula IIa:

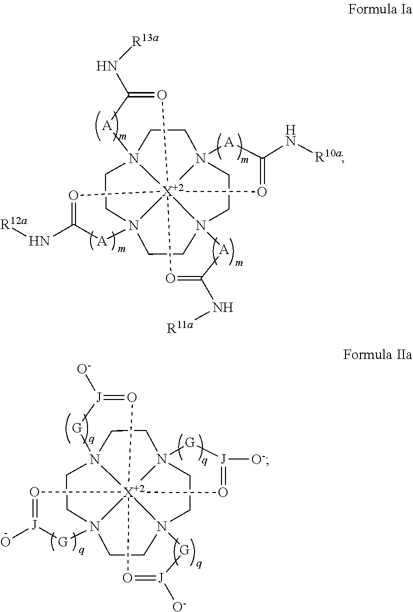

wherein:

each of R^{10a}, R^{11a}, R^{12a}, and R^{13a} is independently selected from the group consisting of

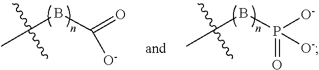

each of A and B is independently CR^{14a}R^{15a};

each G is independently CR^{20a}R^{21a};

each J is independently C or P(O⁻);

each of R^{14a} and R^{15a} is independently selected from the group consisting of hydrogen, C₁-C₆ linear alkyl, and C₃-C₆ branched alkyl, wherein adjacent R^{14a} and R^{15a} groups may bond or fuse to form a C₃-C₁₂ cycloalkyl;

each of R^{20a} and R^{21a} is independently selected from the group consisting of hydrogen, C₁-C₆ linear alkyl, and C₃-C₆ branched alkyl, wherein adjacent R^{20a} and R^{21a} groups may bond or fuse to form a C₃-C₁₂ cycloalkyl;

X is a transition metal selected from the group consisting of Fe, Zn, Co, and Ni, Mn, Au, Pt, Cr, Pd, and Ti; and

each occurrence of m, n, and q is independently an integer from 1-6.

Embodiment 2 provides the compound of embodiment 1, wherein the compound of Formula Ia is

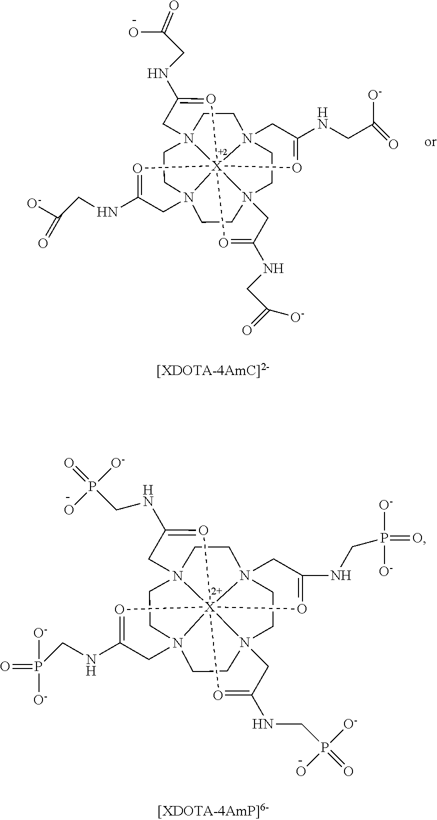

wherein X is selected from the group consisting of Fe, Zn, Co, Ni, Mn, Au, Pt, Cr, Pd, and Ti.

Embodiment 3 provides the compound of any one of embodiments 1-2, wherein X is selected from the group consisting of Fe, Co, and Ni.

Embodiment 4 provides the compound of any one of embodiments 1-3, wherein the agent of Formula IIa is

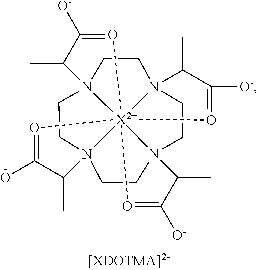

wherein X is selected from the group consisting of Fe, Zn, Co, Ni, Mn, Au, Pt, Cr, Pd, and Ti.

Embodiment 5 provides the compound of any one of embodiments 1-4, wherein X is selected from the group consisting of Fe, Co, and Ni.

Embodiment 6 provides the compound of any one of embodiments 1-5, wherein the compound exhibits high chemical shift sensitivity to temperature and pH.

Embodiment 7 provides the compound of any one of embodiments 1-6, wherein the compound exhibits high chemical shift sensitivity to temperature.

Embodiment 8 provides a method of monitoring at least one of tissue temperature and pH in a patient in need thereof, the method comprising:

administering to the patient a compound of Formula Ia or Formula IIa:

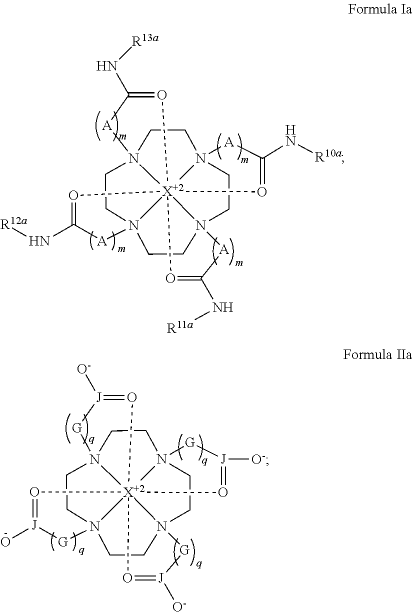

wherein

each of R^{10a}, R^{11a}, R^{12a}, and R^{13a} is independently selected from the group consisting of

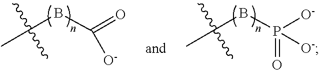

each of A and B is independently CR^{14a}R^{15a};

each G is independently CR^{20a}R^{21a};

each J is independently C or P(O⁻);

each of R^{14a} and R^{15a} is independently selected from the group consisting of hydrogen, C₁-C₆ linear alkyl, and C₃-C₆ branched alkyl, wherein adjacent R^{14a} and R^{15a} groups may bond or fuse to form a C₃-C₁₂ cycloalkyl;

each of R^{20a} and R^{21a} is independently selected from the group consisting of hydrogen, C₁-C₆ linear alkyl, and C₃-C₆ branched alkyl, wherein adjacent R^{20a} and R^{21a} groups may bond or fuse to form a C₃-C₁₂ cycloalkyl;

X is a transition metal selected from the group consisting of Fe, Zn, Co, and Ni, Mn, Au, Pt, Cr, Pd, and Ti; and

each occurrence of m, n, and q is independently an integer from 1-6; and

measuring a change in the chemical shift of one or more protons in the compound.

Embodiment 9 provides the method of embodiment 8, wherein the compound of Formula Ia is

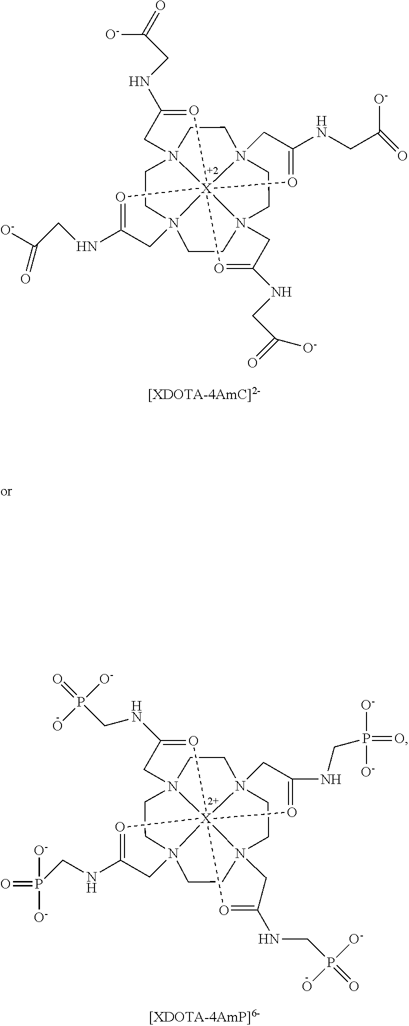

wherein X is selected from the group consisting of Fe, Zn, Co, Ni, Mn, Au, Pt, Cr, Pd, and Ti.

Embodiment 10 provides the method of any one of embodiments 8-9, wherein X is selected from the group consisting of Fe, Co, and Ni.

Embodiment 11 provides the method of any one of embodiments 8-10, wherein the compound of Formula IIa is

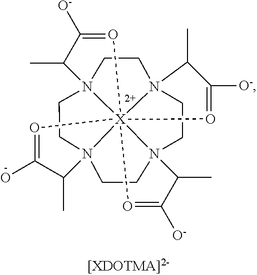

wherein X is selected from the group consisting of Fe, Zn, Co, Ni, Mn, Au, Pt, Cr, Pd, and Ti.

Embodiment 12 provides the method of any one of embodiments 8-11, wherein X from the group consisting of Fe, Co, and Ni.

Embodiment 13 provides the method of any one of embodiments 8-12, wherein the step of administering to the patient the compound is preceded by the step of administering to the patient a chemotherapy drug or pharmaceutical agent.

Embodiment 14 provides the method of any one of embodiments 8-13, wherein the chemotherapy drug or pharmaceutical agent comprises a drug or pharmaceutical agent used to treat glioblastoma.

Embodiment 15 provides the method of any one of embodiments 8-14, wherein the chemotherapy drug or pharmaceutical agent is selected from the group consisting of: everolimus, bevacizumab, sorafenib, carmustine, lomustine, temozolomide, and combinations thereof.

Embodiment 16 provides the method of any one of embodiments 8-15, wherein the chemical shift of the —NH protons are temperature and pH-dependent.

Embodiment 17 provides the method of any one of embodiments 8-16, wherein the chemical shift of the —CH₃ protons are temperature dependent.

Embodiment 18 provides a method of monitoring the efficacy of a cancer treatment in a patient in need thereof, the method comprising:

administering to the patient a chemotherapy drug or pharmaceutical agent;

administering to the patient, at the site of a tumor, a compound of Formula Ia or Formula IIa:

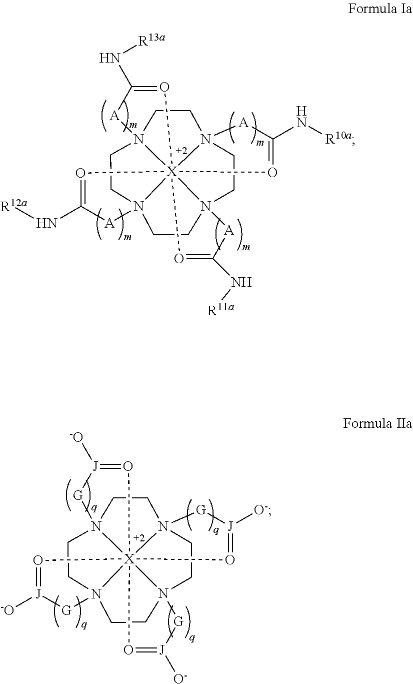

wherein:

each of R^{10a}, R^{11a}, R^{12a}, and R^{13a} is independently selected from the group consisting of

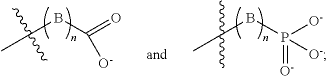

each of A and B is independently CR^{14a}R^{15a};

each G is independently CR^{20a}R^{21a};

each J is independently C or P(O⁻);

each of R^{14a} and R^{15a} is independently selected from the group consisting of hydrogen, C₁-C₆ linear alkyl, and C₃-C₆ branched alkyl, wherein adjacent R^{14a} and R^{15a} groups may bond or fuse to form a C₃-C₁₂ cycloalkyl;

each of R^{20a} and R^{21a} is independently selected from the group consisting of hydrogen, C₁-C₆ linear alkyl, and C₃-C₆ branched alkyl, wherein adjacent R^{20a} and R^{21a} groups may bond or fuse to form a C₃-C₁₂ cycloalkyl;

X is a transition metal selected from the group consisting of Fe, Zn, Co, and Ni, Mn, Au, Pt, Cr, Pd, and Ti; and

each occurrence of m, n, and q is independently an integer from 1-6; and

measuring a change in the chemical shift of one or more protons in the compound.

Embodiment 19 provides the method of embodiment 18, wherein the step of measuring a change in the chemical shift of one or more protons in the compound further comprises the step of mapping the extracellular pH inside and outside of the tumor.

Embodiment 20 provides the method of any one of embodiments 18-19, wherein the step of measuring a change in the chemical shift of one or more protons in the compound further comprises the step of mapping the temperature inside and outside of the tumor.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the disclosure. The appended claims are intended to be construed to include all such embodiments and equivalent variations.

What is claimed is:

1. A compound of Formula Ia or Formula IIa: 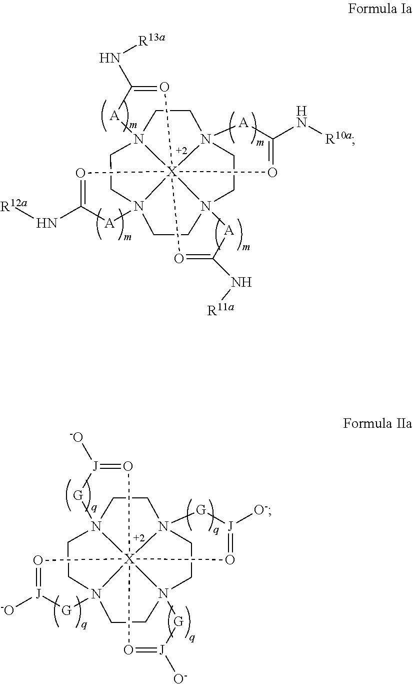
wherein:
each of R^{10a}, R^{11a}, R^{12a}, and R^{13a} is independently selected from the group consisting of 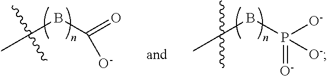
each of A and B is independently CR^{14a}R^{15a};
each G is independently CR^{20a}R^{21a};
each J is independently C or P(O⁻);
each of R^{14a} and R^{15a} is independently selected from the group consisting of hydrogen, C₁-C₆ linear alkyl, and C₃-C₆ branched alkyl, wherein adjacent R^{14a} and R^{15a} groups may bond or fuse to form a C₃-C₁₂ cycloalkyl;
each of R^{20a} and R^{21a} is independently selected from the group consisting of hydrogen, C₁-C₆ linear alkyl, and C₃-C₆ branched alkyl, wherein adjacent R^{20a} and R^{21a} groups may bond or fuse to form a C₃-C₁₂ cycloalkyl;
X is a transition metal selected from the group consisting of Fe, Zn, Co, and Ni, Mn, Au, Pt, Cr, Pd, and Ti; and
each occurrence of m, n, and q is independently an integer from 1-6.

2. The compound of claim 1, wherein the compound of Formula Ia is 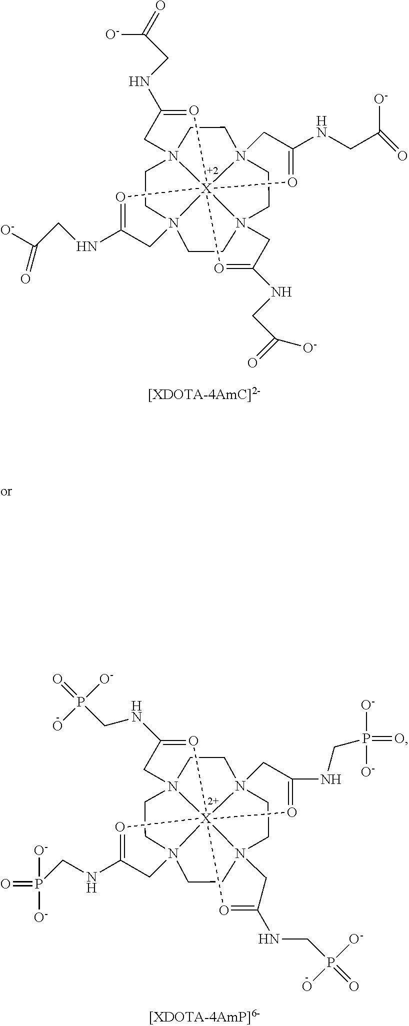
wherein X is selected from the group consisting of Fe, Zn, Co, Ni, Mn, Au, Pt, Cr, Pd, and Ti.

3. The compound of claim 2, wherein X is selected from the group consisting of Fe, Co, and Ni.

4. The compound of claim 1, wherein the agent of Formula IIa is 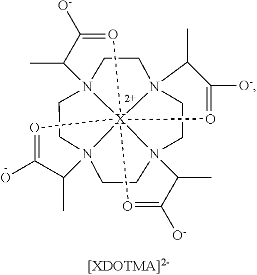
wherein X is selected from the group consisting of Fe, Zn, Co, Ni, Mn, Au, Pt, Cr, Pd, and Ti.

5. The compound of claim 4, wherein X is selected from the group consisting of Fe, Co, and Ni.

6. The compound of claim 2, wherein the compound exhibits high chemical shift sensitivity to temperature and pH.

7. The compound of claim 4, wherein the compound exhibits high chemical shift sensitivity to temperature.

8. A method of monitoring at least one of tissue temperature and pH in a patient in need thereof, the method comprising:
administering to the patient a compound of Formula Ia or Formula IIa: 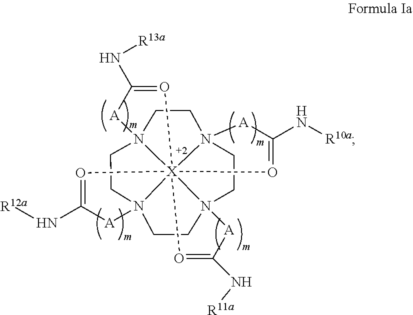 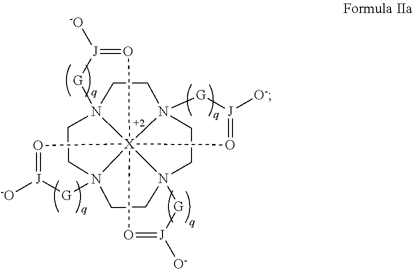
wherein
each of R^{10a}, R^{11a}, R^{12a}, and R^{13a} is independently selected from the group consisting of 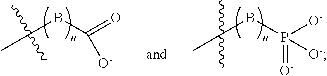
each of A and B is independently CR^{14a}R^{15a};
each G is independently CR^{20a}R^{21a};
each J is independently C or P(O⁻);
each of R^{14a} and R^{15a} is independently selected from the group consisting of hydrogen, C₁-C₆ linear alkyl, and C₃-C₆ branched alkyl, wherein adjacent R^{14a} and R^{15a} groups may bond or fuse to form a C₃-C₁₂ cycloalkyl;
each of R^{20a} and R^{21a} is independently selected from the group consisting of hydrogen, C₁-C₆ linear alkyl, and C₃-C₆ branched alkyl, wherein adjacent R^{20a} and R^{21a} groups may bond or fuse to form a C₃-C₁₂ cycloalkyl;
X is a transition metal selected from the group consisting of Fe, Zn, Co, and Ni, Mn, Au, Pt, Cr, Pd, and Ti; and
each occurrence of m, n, and q is independently an integer from 1-6; and
measuring a change in the chemical shift of one or more protons in the compound.

9. The method of claim 8, wherein the compound of Formula Ia is 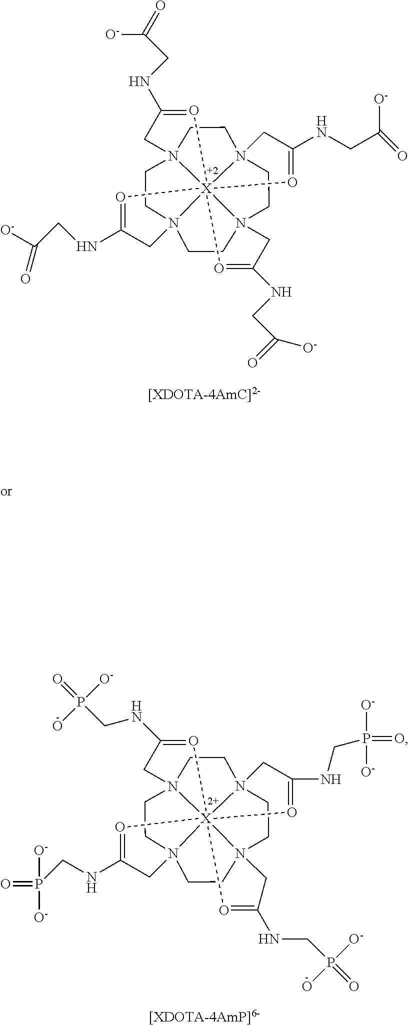
wherein X is selected from the group consisting of Fe, Zn, Co, Ni, Mn, Au, Pt, Cr, Pd, and Ti.

10. The method of claim 9, wherein X is selected from the group consisting of Fe, Co, and Ni.

11. The method of claim 8, wherein the compound of Formula IIa is 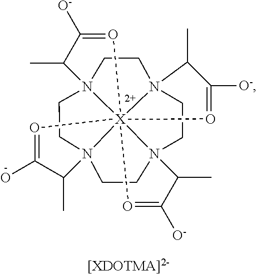
wherein X is selected from the group consisting of Fe, Zn, Co, Ni, Mn, Au, Pt, Cr, Pd, and Ti.

12. The method of claim 11, wherein X from the group consisting of Fe, Co, and Ni.

13. The method of claim 8, wherein the step of administering to the patient the compound is preceded by the step of administering to the patient a chemotherapy drug or pharmaceutical agent.

14. The method of claim 13, wherein the chemotherapy drug or pharmaceutical agent comprises a drug or pharmaceutical agent used to treat glioblastoma.

15. The method of claim 14, wherein the chemotherapy drug or pharmaceutical agent is selected from the group consisting of: everolimus, bevacizumab, sorafenib, carmustine, lomustine, temozolomide, and combinations thereof.

16. The method of claim 9, wherein the chemical shift of the —NH protons are temperature and pH-dependent.

17. The method of claim 11, wherein the chemical shift of the —CH₃ protons are temperature dependent.

18. A method of monitoring the efficacy of a cancer treatment in a patient in need thereof, the method comprising:
administering to the patient a chemotherapy drug or pharmaceutical agent;
administering to the patient, at the site of a tumor, a compound of Formula Ia or Formula IIa: 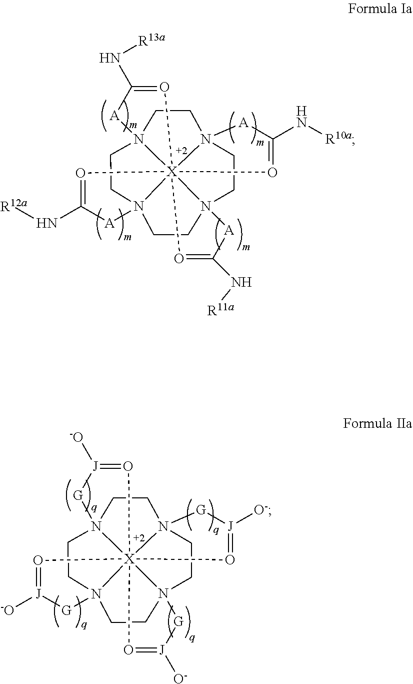
wherein:
each of R^{10a}, R^{11a}, R^{12a}, and R^{13a} is independently selected from the group consisting of 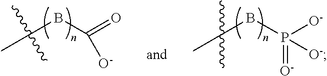
each of A and B is independently CR^{14a}R^{15a};
each G is independently CR^{20a}R^{21a};
each J is independently C or P(O⁻);
each of R^{14a} and R^{15a} is independently selected from the group consisting of hydrogen, C₁-C₆ linear alkyl, and C₃-C₆ branched alkyl, wherein adjacent R^{14a} and R^{15a} groups may bond or fuse to form a C₃-C₁₂ cycloalkyl;
each of R^{20a} and R^{21a} is independently selected from the group consisting of hydrogen, C₁-C₆ linear alkyl, and C₃-C₆ branched alkyl, wherein adjacent R^{20a} and R^{21a} groups may bond or fuse to form a C₃-C₁₂ cycloalkyl;
X is a transition metal selected from the group consisting of Fe, Zn, Co, and Ni, Mn, Au, Pt, Cr, Pd, and Ti; and
each occurrence of m, n, and q is independently an integer from 1-6; and
measuring a change in the chemical shift of one or more protons in the compound.

19. The method of claim 18, wherein the step of measuring a change in the chemical shift of one or more protons in the compound further comprises the step of mapping the extracellular pH inside and outside of the tumor.

20. The method of claim 18, wherein the step of measuring a change in the chemical shift of one or more protons in the compound further comprises the step of mapping the temperature inside and outside of the tumor.
